SEQUENCE LISTING (31 sequences)

SEQ ID NO: 1 - protein, 159 aa, Human herpesvirus 6
MGYEEKVSATGKTRLKILACLIVLILAAAITMLTLEIISNQKRTTTDLEAVTVALKHVSTSLASCTESTTSVHTDSVTSQPTKNKESRKKIEGKSPSWVQALTTASGIILLFCIMMIFITCSWTTEKDTEKSEVQSYASSVETLDSLNEAIIPKTEMNV

SEQ ID NO: 2 - DNA, 480 bp, Human herpesvirus 6
atgggatatgaagaaaaagtgtcagctactggaaagactcgtttaaagatactggcatgtctgatcgttttaatactagctgcggcaataactatgttaacgctggaaattatatcgaaccaaaaacgtaccactactgatctcgaagctgtgactgtggcgctgaagcatgtaagcacatctcttgccagctgcactgaatccactacttctgtacataccgattctgtgacgagccaacccacgaaaaacaaagaatcgaggaaaaaaattgaagggaaatctccaagttgggttcaggctttaactacagcatctggaattatcctactgttttgtataatgatgatattcattacatgttcctggaccacagaaaaagatacagagaagagtgaagtgcaatcttatgcttcttcagtagagactttagactctttaaatgaggctattataccgaaaactgaaatgaatgtgtaa

SEQ ID NO: 3 - DNA, 1795 bp, Human herpesvirus 6
aggctctgctggaggctctgctggaggccttgctgaaggctctgctggaggccctgctggaggtcttgctggaggctctgctggaggctctgctggaggctctgctggaggctctgctggaggctctgctggaggctctgctggaggctctgtcagagacctcggtgaaagttttactcagaggtttatcagagttttcgccattagtttggttagaagtttcagatttattttcggtggaactgcagttaggtttcatgtcagtacattcatcaccgttagaagtgctattcatggtgctgttgccactgttggatttgttaaaagcagtaaatgagctaggattggaatgactccgaatagctaataaatttgagcattttcttcgaatggatcataatcagagggatagccatctaatttaaagacttccattttatcactgttgcaatcacttctaatggagtatctggatacattttttctacatctttttcatcccctccaacatggatctgtgcagcgttaataagccagcggagttaattaaatcgtcttccatgttagacagttcctgtttcatggcagccttcactgatgcaccaatactttggatgcaagtgccaacggactgagctaggatgtaaaagaagatattctaattttgaattcttcagatgctccttcttccacattactggaataggacacattcttggaagcgatgtcgttggaagactctgggatgaaaagatcacaggcttccagttctggaaaaagcaggctttcaaaggacacatcacacttgagactctcttccaatatttctttgatggattcttccaccactggatcgggatggtagctatatatactatataaggagattaccaccaccacctctttctttgcagagattattctctgcttgaaaatctgtaacactgatcatgatgggatatgaagaaaaagtgtcagctactggaaagactcgtttaaagatactggcatgtctgatcgttttaatactagctgcggcaataactatgttaacgctggaaattatatcgaaccaaaaacgtaccactactgatctcgaagctgtgactgtggcgctgaagcatgtaagcacatctcttgccagctgcactgaatccactacttctgtacataccgattctgtgacgagccaacccacgaaaaacaaagaatcgaggaaaaaaattgaagggaaatctccaagttgggttcaggctttaactacagcatctggaattatcctactgttttgtataatgatgatattcattacatgtccctggaccacagaaaaagatacagagaagagtgaagtgcaatcttatgctccttcagtagagactttagaccctttaaatgaggctattataccgaaaactgaaatgaatgtgtaatgtctgtatttttctttacagagatgtacggagagtttatatttggggaaaatacctgactgttctgcctatatgcgaatgttaaagtatgtataatataaattcttaccttttaagagtgattcaaggtggaggtttctttggagattgattccaggtggtggtttcgggtgcaatcaatctttcttctgggcgggaagaaaatccagcaatccaataattgatgggatgtaatcaatgtcacaaatctgtaagattaaatgtgaacagtataaattctttcgtgcttatcaaattacaattatgcgcatgaaaatatcattaaattgttttaaacattcttaaaaaaaaaaaaaaaaaaa

SEQ ID NO: 4 - DNA, 432 bp, Pleurotus cornucopiae
  CDS (1)..(432)
atgaaagacgtccaatctctcctcaccggaacctggtacaatgaactcggctcaacaatgaatttgactgcaaataaagacggttcgctcaccggaacgtaccactccaacgtcggcgaggttcccccaacttatcacctttctggccggtacaacctccagcccccctcgggtcaaggcgttactctgggatgggcggtgtctttcgaaaacactagtgcgaatgttcattctgtctcaacatggagcgggcagtacttctctgaacccgccgaggtgatcctcacccagtggctgttgtcaaggagctctgagcgcgaagatttgtggcagtccacccatgtggggcatgatgagttcagcaagacaaagccaaccaaggagaagattgcccaggctcaactccttcgtcgcgggttgaagttcgagtga

SEQ ID NO: 5 - protein, 143 aa, Pleurotus cornucopiae
MKDVQSLLTGTWYNELGSTMNLTANKDGSLTGTYHSNVGEVPPTYHLSGRYNLQPPSGQGVTLGWAVSFENTSANVHSVSTWSGQYFSEPAEVILTQWLLSRSSEREDLWQSTHVGHDEFSKTKPTKEKIAQAQLLRRGLKFE

SEQ ID NO: 6 - DNA, 426 bp, Pleurotus cornucopiae
  CDS (1)..(426)
atgtcagacgttcaatcttcactcaccggaacctggtacaatgaactcaactccaagatggaattgactgcaaacaaagacggtactctcactggaaagtacctctccaaagttggggatgtctacgtgccctacccactctctggtcgctataacctccaaccccccgcgggacaaggcgtcgctcttgggtgggcggtatcctgggagaacagtaaaattcattccgctacgacatggagcggacagttcttctctgagtcgtctccagtgattcttactcagtggttgttgtcatcgagcactgcgcgtggggacgtatgggaatccacacttgtggggaatgattcgtttacaaagacggcgccgactgagcagcagatcgctcatgctcaactccattgtcgcgcaccgaggttgaagtaa

SEQ ID NO: 7 - protein, 141 aa, Pleurotus cornucopiae
MSDVQSSLTGTWYNELNSKMELTANKDGTLTGKYLSKVGDVYVPYPLSGRYNLQPPAGQGVALGWAVSWENSKIHSATTWSGQFFSESSPVILTQWLLSSSTARGDVWESTLVGNDSFTKTAPTEQQIAHAQLHCRAPRLK

SEQ ID NO: 8 - protein, 5 aa, Artificial Sequence
  Synthetic peptide linker
GGGGS

SEQ ID NO: 9 - protein, 5 aa, Artificial Sequence
  Synthetic peptide linker
GGSGG

SEQ ID NO: 10 - protein, 5 aa, Artificial Sequence
  Synthetic peptide linker
GASAG

SEQ ID NO: 11 - protein, 5 aa, Artificial Sequence
  Synthetic peptide linker
GSGAA

SEQ ID NO: 12 - protein, 5 aa, Artificial Sequence
  Synthetic peptide linker
GSGSA

SEQ ID NO: 13 - protein, 6 aa, Artificial Sequence
  Synthetic peptide linker
GGGGSG

SEQ ID NO: 14 - protein, 7 aa, Artificial Sequence
  Synthetic peptide linker
GGGSGGS

SEQ ID NO: 15 - protein, 8 aa, Artificial Sequence
  Synthetic peptide linker
GGSGGGGS

SEQ ID NO: 16 - protein, 9 aa, Artificial Sequence
  Synthetic peptide linker
AAAAGSGAA

SEQ ID NO: 17 - protein, 15 aa, Artificial Sequence
  Synthetic peptide linker
GGGGSGGGGSGGGGS

SEQ ID NO: 18 - protein, 25 aa, Artificial Sequence
  Synthetic peptide linker
GGGGSGGGGSGGGGSGGGGSGGGGS

SEQ ID NO: 19 - DNA, 117 bp, Artificial Sequence
  Synthetic primer SITH1C-5xlink-TM2N-F
ccgaaaactgaaatgaatgtgggtggcggtggcagcggtggcggtggcagcggtggcggtggcagcggtggcggtggcagcggtggcggtggcagctcagacgttcaatcttcactc

SEQ ID NO: 20 - DNA, 117 bp, Artificial Sequence
  Synthetic primer SITH1C-5xlink-TM2N-R
gagtgaagattgaacgtctgagctgccaccgccaccgctgccaccgccaccgctgccaccgccaccgctgccaccgccaccgctgccaccgccacccacattcatttcagttttcgg

SEQ ID NO: 21 - DNA, 29 bp, Artificial Sequence
  Synthetic primer SITH1C-5′ EcoRI-F
aaagaattcggatatgaagaaaaagtgtc

SEQ ID NO: 22 - DNA, 28 bp, Artificial Sequence
  Synthetic primer TM2CtermBam
tttggatccttacttcaacctcggtgcg

SEQ ID NO: 23 - DNA, 981 bp, Artificial Sequence
  DNA encoding fusion protein SITH-1-TM2
atggaattcggatatgaagaaaaagtgtcagctactggaaagactcgtttaaagatactggcatgtctgatcgttttaatactagctgcggcaataactatgttaacgctggaaattatatcgaaccaaaaacgtaccactactgatctcgaagctgtgactgtggcgctgaagcatgtaagcacatctcttgccagctgcactgaatccactacttctgtacataccgattctgtgacgagccaacccacgaaaaacaaagaatcgaggaaaaaaattgaagggaaatctccaagttgggttcaggctttaactacagcatctggaattatcctactgttttgtataatgatgatattcattacatgttcctggaccacagaaaaagatacagagaagagtgaagtgcaatcttatgcttcttcagtagagactttagactctttaaatgaggctattataccgaaaactgaaatgaatgtgggtggcggtggcagcggtggcggtggcagcggtggcggtggcagcggtggcggtggcagcggtggcggtggcagctcagacgttcaatcttcactcaccggaacctggtacaatgaactcaactccaagatggaattgactgcaaacaaagacggtactctcactggaaagtacctctccaaagttggggatgtctacgtgccctacccactctctggtcgctataacctccaaccccccgcgggacaaggcgtcgctcttgggtgggcggtatcctgggagaacagtaaaattcattccgctacgacatggagcggacagttcttctctgagtcgtctccagtgattcttactcagtggttgttgtcatcgagcactgcgcgtggggacgtatgggaatccacacttgtggggaatgattcgtttacaaagacggcgccgactgagcagcagatcgctcatgctcaactccattgtcgcgcaccgaggttgaagtaa

SEQ ID NO: 24 - protein, 326 aa, Artificial Sequence
  Synthetic Construct Fusion protein SITH-1-TM2
MEFGYEEKVSATGKTRLKILACLIVLILAAAITMLTLEIISNQKRTTTDLEAVTVALKHVSTSLASCTESTTSVHTDSVTSQPTKNKESRKKIEGKSPSWVQALTTASGIILLFCIMMIFITCSWTTEKDTEKSEVQSYASSVETLDSLNEAIIPKTEMNVGGGGSGGGGSGGGGSGGGGSGGGGSSDVQSSLTGTWYNELNSKMELTANKDGTLTGKYLSKVGDVYVPYPLSGRYNLQPPAGQGVALGWAVSWENSKIHSATTWSGQFFSESSPVILTQWLLSSSTARGDVWESTLVGNDSFTKTAPTEQQIAHAQLHCRAPRLK

SEQ ID NO: 25 - DNA, 29 bp, Artificial Sequence
  Synthetic primer HarpinNtermEcoRI-F
aaagaattcatgcagagtctcagtcttaa

SEQ ID NO: 26 - DNA, 114 bp, Artificial Sequence
  Synthetic primer HarpinC-5xlink-TM2N-R
gtgaagattgaacgtctgagctgccaccgccaccgctgccaccgccaccgctgccaccgccaccgctgccaccgccaccgctgccaccgccaccggctgcagcctgattgcggg

SEQ ID NO: 27 - DNA, 114 bp, Artificial Sequence
  Synthetic primer HarpinC-5xlink-TM2N-F
cccgcaatcaggctgcagccggtggcggtggcagcggtggcggtggcagcggtggcggtggcagcggtggcggtggcagcggtggcggtggcagctcagacgttcaatcttcac

SEQ ID NO: 28 - DNA, 1029 bp, Pseudomonas syringae
  CDS (1)..(1029)
atgcagagtctcagtcttaacagcagctcgctgcaaaccccggcaatggcccttgtcctggtacgtcctgaaaccgagacgactggcgccagtacgtcgagcaaggcgcttcaggaagttgtcgtgaagctggccgaggaactgatgcgcaatggtcaactcgacgacagctcgccattgggcaaactgctggccaagtcgatggccgcggatggcaaggcaggcggcggtatcgaggatgtcatcgctgcgctggacaagctgattcatgaaaagctgggtgacaacttcggcgcgtctgcggacaacgcctcgggtaccggacagcaggacctgatgactcaggtgctcagtggcctggccaagtctatgctcgatgatcttctgaccaagcaggatggcggggcaagcttctccgaagacgatatgccgatgctgaacaagatcgcgcagttcatggatgacaatcccgcacagtttcccaagccggactcgggttcctgggtgaacgaactcaaggaagacaacttccttgatggcgacgaaacggctgcgttccgctcggcactcgacatcattggccagcaactgggtaatcagcagagtggcgctggcggtctggcggggacgggtggaggtctgggcactccgagcagtttttctaacaactcgtccgtgacgggtgatccgctgatcgacgccaataccggtcccggtgacagcggcaatagcagtggtgaggcggggcaactgatcggcgagcttatcgaccgtggcctgcaatcggtattggccggtggtggactgggcacacccgtaaacaccccgcagaccggtacggcggcgaatggcggacagtccgctcaggatcttgaccagttgctgggcggcttgctgctcaagggccttgaagcgacgctcaaggatgccggtcaaaccgctaccgacgtgcagtcgagcgctgcgcaaatcgccaccttgctggtcagtacgctgctgcaaggcacccgcaatcaggctgcagcctga

SEQ ID NO: 29 - protein, 342 aa, Pseudomonas syringae
MQSLSLNSSSLQTPAMALVLVRPETETTGASTSSKALQEVVVKLAEELMRNGQLDDSSPLGKLLAKSMAADGKAGGGIEDVIAALDKLIHEKLGDNFGASADNASGTGQQDLMTQVLSGLAKSMLDDLLTKQDGGASFSEDDMPMLNKIAQFMDDNPAQFPKPDSGSWVNELKEDNFLDGDETAAFRSALDIIGQQLGNQQSGAGGLAGTGGGLGTPSSFSNNSSVTGDPLIDANTGPGDSGNSSGEAGQLIGELIDRGLQSVLAGGGLGTPVNTPQTGTAANGGQSAQDLDQLLGGLLLKGLEATLKDAGQTATDVQSSAAQIATLLVSTLLQGTRNQAAA

SEQ ID NO: 30 - DNA, 1524 bp, Artificial Sequence
  DNA encoding fusion protein Harpin-TM2
atgcagagtctcagtcttaacagcagctcgctgcaaaccccggcaatggcccttgtcctggtacgtcctgaaaccgagacgactggcgccagtacgtcgagcaaggcgcttcaggaagttgtcgtgaagctggccgaggaactgatgcgcaatggtcaactcgacgacagctcgccattgggcaaactgctggccaagtcgatggccgcggatggcaaggcaggcggcggtatcgaggatgtcatcgctgcgctggacaagctgattcatgaaaagctgggtgacaacttcggcgcgtctgcggacaacgcctcgggtaccggacagcaggacctgatgactcaggtgctcagtggcctggccaagtctatgctcgatgatcttctgaccaagcaggatggcggggcaagcttctccgaagacgatatgccgatgctgaacaagatcgcgcagttcatggatgacaatcccgcacagtttcccaagccggactcgggttcctgggtgaacgaactcaaggaagacaacttccttgatggcgacgaaacggctgcgttccgctcggcactcgacatcattggccagcaactgggtaatcagcagagtggcgctggcggtctggcggggacgggtggaggtctgggcactccgagcagtttttctaacaactcgtccgtgacgggtgatccgctgatcgacgccaataccggtcccggtgacagcggcaatagcagtggtgaggcggggcaactgatcggcgagcttatcgaccgtggcctgcaatcggtattggccggtggtggactgggcacacccgtaaacaccccgcagaccggtacggcggcgaatggcggacagtccgctcaggatcttgaccagttgctgggcggcttgctgctcaagggccttgaagcgacgctcaaggatgccggtcaaaccgctaccgacgtgcagtcgagcgctgcgcaaatcgccaccttgctggtcagtacgctgctgcaaggcacccgcaatcaggctgcagccggtggcggtggcagcggtggcggtggcagcggtggcggtggcagcggtggcggtggcagcggtggcggtggcagctcagacgttcaatcttcactcaccggaacctggtacaatgaactcaactccaagatggaattgactgcaaacaaagacggtactctcactggaaagtacctctccaaagttggggatgtctacgtgccctacccactctctggtcgctataacctccaaccccccgcgggacaaggcgtcgctcttgggtgggcggtatcctgggagaacagtaaaattcattccgctacgacatggagcggacagttcttctctgagtcgtctccagtgattcttactcagtggttgttgtcatcgagcactgcgcgtggggacgtatgggaatccacacttgtggggaatgattcgtttacaaagacggcgccgactgagcagcagatcgctcatgctcaactccattgtcgcgcaccgaggttgaagtaa

SEQ ID NO: 31 - protein, 507 aa, Artificial Sequence
  Synthetic Construct Fusion protein Harpin-TM2
MQSLSLNSSSLQTPAMALVLVRPETETTGASTSSKALQEVVVKLAEELMRNGQLDDSSPLGKLLAKSMAADGKAGGGIEDVIAALDKLIHEKLGDNFGASADNASGTGQQDLMTQVLSGLAKSMLDDLLTKQDGGASFSEDDMPMLNKIAQFMDDNPAQFPKPDSGSWVNELKEDNFLDGDETAAFRSALDIIGQQLGNQQSGAGGLAGTGGGLGTPSSFSNNSSVTGDPLIDANTGPGDSGNSSGEAGQLIGELIDRGLQSVLAGGGLGTPVNTPQTGTAANGGQSAQDLDQLLGGLLLKGLEATLKDAGQTATDVQSSAAQIATLLVSTLLQGTRNQAAAGGGGSGGGGSGGGGSGGGGSGGGGSSDVQSSLTGTWYNELNSKMELTANKDGTLTGKYLSKVGDVYVPYPLSGRYNLQPPAGQGVALGWAVSWENSKIHSATTWSGQFFSESSPVILTQWLLSSSTARGDVWESTLVGNDSFTKTAPTEQQIAHAQLHCRAPRLK

(19) United States
(12) Patent Application Publication
(10) Pub. No.: US 2012/0064543 A1
(43) Pub. Date: Mar. 15, 2012

(54) METHOD FOR DETECTING SUBSTANCE IN BIOLOGICAL SAMPLE

(75) Inventors: Yoshimitsu Takakura, Shizuoka (JP); Naomi Oka, Shizuoka (JP); Kazuhiro Kondo, Tokyo (JP)

(73) Assignees: VIRUS IKAGAKU KENKYUSHO INC., Toyonaka-shi, Osaka (JP); JAPAN TOBACCO INC., Tokyo (JP)

(21) Appl. No.: 13/254,128

(22) PCT Filed: Mar. 2, 2010
(86) PCT No.: PCT/JP2010/053359

§ 371 Date: Nov. 23, 2011

(30) Foreign Application Priority Data
Mar. 2, 2009 (JP) 2009-048661

(51) Int. Cl. G01N 33/82; C07K 19/00

(52) U.S. Cl. 435/7.5; 436/501; 530/408

(57) ABSTRACT

The present invention provides a method for detecting a substance in a biological sample, a carrier for using in the method, and a kit. The method of the present invention according to an embodiment includes:
1) providing a carrier on which biotin is bound and providing a fusion protein between a protein that specifically binds to a substance to be detected and a biotin-binding protein;
2) binding the fusion protein to the carrier provided in step 1) through binding between biotin and the biotin-binding protein to produce a fusion protein-bound carrier;
3) mixing
(a) a biological sample, and
(b-i) a cell homogenate extract prepared from cells of the same species as the host cells used for expressing the fusion protein in step 1), and a biotin-binding protein, or
(b-ii) a cell homogenate extract prepared from cells of the same species as the host cells used for expressing the fusion protein in step 1), and genetically engineered to express a biotin-binding protein, and adding the mixture to the fusion protein-bound carrier produced in step 2); and
4) detecting a substance that has bound to the protein that specifically binds to a substance to be detected in the fusion protein.

### TECHNICAL FIELD

The present invention relates to a method for qualitatively and/or quantitatively detecting a substance in a biological sample. In particular, the method of the present invention can detect even a trace amount of substance that is present in a biological sample and that cannot be readily detected in usual manner.

### BACKGROUND ART

Method for Detecting a Substance to be Detected Utilizing a Carrier on Which a Protein that can Specifically Binds to the Substance is Immobilized by Hydrophobic Bonding, Covalent Bonding, etc.

In order to detect a substance in a biological sample, a method utilizing a substance that specifically binds to the former substance (substance to be detected) has been widely used. As the substances that specifically bind to the substance to be detected, for example, antibodies or other proteins are usually used. These substances can be immobilized on a carrier such as a microplate, microbeads, or a sensor chip. For example, hydrophobic bonding and covalent bonding are known as general means for the immobilization.

In the “hydrophobic bonding”, a carrier and a protein that specifically binds to a substance to be detected (hereinafter, may be referred to as “specific protein”) are bound to each other by interaction between the hydrophobic surface of the carrier and the hydrophobic moiety of the specific protein. This is convenient from the point of not needing specific reagents. However, such binding is usually weak. In the case where the hydrophobic bonding is applied to, for example, enzyme-linked immunosorbent assay (ELISA), the protein is detached from the carrier during, for example, a washing procedure after binding in many cases. Furthermore, in the case where a specific protein is bound to a carrier by hydrophobic bonding, the function of the protein may be lost completely or partially in many cases.

The “covalent bonding” utilizes interaction between functional groups (e.g., amino groups) of a specific protein and functional groups (e.g., carboxyl group) provided on the surface of the carrier, and is strong. However, after a specific protein is bound to a carrier by covalent bonding, the function of the protein is lost completely or partially in many cases, like the hydrophobic bonding.

In addition to the hydrophobic bonding and the covalent bonding, known is a method for fusing a plurality of histidine molecules to terminals of protein molecules and binding the fusion protein having the histidine tags to, for example, a base plate, such as a protein chip, having a surface provided with nickel. The interaction between the histidine tags and nickel ions is, however, not very strong, and nickel ions are known to non-specifically bind to a variety of biological molecules.

In such a specific binding assay system using a solid phase on which a protein that specifically binds to a substance to be detected is bound by hydrophobic bonding or covalent bonding, non-specific binding, which causes background signals and thus should be reduced, is generally a severe problem. In order to solve this problem, the following methods have been proposed for example: a method of adding an extract of a bacterium component to a reagent for detection (JP No. S59-99257 A (1984)); a method of adding a culture component of host cells containing a vector of the same species as that used in production of a recombinant protein capable of specifically binding to a substance to be detected and the vector not containing the gene encoding the protein to a sample (JP No. H8-43392 A (1996)); and a method of heat-treating an aqueous extract from cells of the same species as that producing a recombinant protein capable of specifically binding to a substance to be detected, and the cell not containing this protein, and then adding water-soluble fraction of the heated aqueous extract to a sample (JP No. 2004-301646 A). These methods show some effects on inhibition of non-specific binding.

Detection of Substance Utilizing Avidin-Biotin Binding

Avidin is a glycoprotein derived from egg white and extremely strongly binds to biotin (vitamin H). The interaction between avidin and biotin is one of the strongest non-covalent bonds (Green, (1975), Adv Protein Chem; 29: 85-133). On the other hand, streptavidin is an avidin-like protein derived from Streptomyces avidinii and also strongly binds to biotin. The interaction of (strept)avidin-biotin, because of its high acting force, has been widely applied, for example, for detection of antigens and antibodies in the fields of molecular biology and biochemistry (Green, (1990), Methods Enzymol, 184: 51-67).

A method has been proposed for binding a protein to a carrier by the biotin-binding ability of avidin or streptavidin. That is, the method involves binding (strept)avidin to a base plate such as a microplate by covalent bonding or hydrophobic bonding and further binding to a biotinylated protein to immobilize the protein.

A technology of immobilizing base plate-biotin-avidin-biotin-desired protein in this order is also reported in which an avidin protein is bound to a base plate provided with biotin by avidin-biotin binding and then a biotinylated desired protein thereto at another biotin pocket of the avidin (JP No. H4-236353 A (1992)). A substance to be detected can be detected using a plate on which a specific protein is immobilized by such a method.

The assay utilizing the avidin-biotin binding has also a big problem with a large background signal, like the assay using a solid phase on which a protein that specifically binds to a substance to be detected is bound by, for example, hydrophobic bonding or covalent bonding. Countermeasures have been proposed for solving this problem are, for example: a method in which a sample is put into contact with a solid phase to which inactivated (strept)avidin is bound and then contact with a solid phase to which active (strept)avidin is bound (JP No. H8-114590 A (1996)); a method in which a biotinylated substance is bound to a avidin-bound solid phase, and then this is put into contact with a conjugate of polyethylene glycol and biotin (JP No. H11-211727 A (1999)); and a method in which a biotin-containing solution is put into contact with a solid phase (JP No. 2002-48794 A), in addition to the above-mentioned methods for preventing non-specific binding. Unfortunately, all the methods exhibit insufficient practical advantages.

In the past, fusion proteins have been produced using avidin or streptavidin in order to label a protein or to use as a diagnostic marker or a targeted cell-specific factor (Airenne et al., (1999), Biomol Eng, 16: 87-92). Application of these fusion proteins, in particular, fusion proteins of avidin or streptavidin and antibodies such as scFv, Fab fragment, or IgG to agents for specifically targeting, for example, cancer cells has been studied. In addition, an idea of a column is described on which scFv is immobilized through avidin-biotin binding using a fusion protein between streptavidin and scFv (Kiprivanov et al., (1995), Hum Antib Hybrid, 6: 93-101; Dubel et al., (1995), J Immunol Methods, 178: 201-209). However, no example is known on immobilization using a biotin-binding protein to detect a substance to be detected in a biological sample. There is a report on an example in which ELISA is performed by immobilizing a streptavidin fusion protein on a biotinylated plate and using an antibody for the fusion protein as the antigen (WO2002/046395), but this merely demonstrates that a streptavidin fusion protein can be immobilized on a carrier without losing its activity.

### CITATION LIST

### Patent Document

Patent Document 1: JP No. S59-99257 A

Patent Document 2: JP No. H8-43392 A

Patent Document 3: JP No. 2004-301646 A

Patent Document 4: JP No. H8-114590 A

Patent Document 5: JP No. H11-211727 A

Patent Document 6: JP No. 2002-48794 A

Patent Document 7: WO2002/046395

Patent Document 8: WO2002/072817

### Non-Patent Document

Non-Patent Document 1: Green, (1975), Adv Protein Chem, 29: 85-133

Non-Patent Document 2: Green, (1990), Methods Enzymol, 184: 51-67

Non-Patent Document 3: Airenne et al., (1999), Biomol Eng, 16: 87-92

Non-Patent Document 4: Kiprivanov et al., (1995), Hum Antib Hybrid, 6: 93-101

Non-Patent Document 5: Dubel et al., (1995), J Immunol Methods, 178: 201-209

Non-Patent Document 6: Takakura et al., (2009), FEBS J, 276: 1383-1397

### SUMMARY OF INVENTION

### Technical Problem

It is an object of the present invention to provide a method for qualitatively and/or quantitatively detecting a substance in a biological sample.

Specifically, it is an object of the present invention to provide a method for qualitatively and/or quantitatively detecting a trace amount of substance that is present in a biological sample while reducing the background signal level.

### Solution to Problem

The inventors have diligently studied and, as a result, have developed a system in which a fusion protein between a protein that specifically binds to a substance to be detected and a biotin-binding protein is immobilized on a carrier through binding between biotin and the biotin-binding protein. Furthermore, the inventors have discovered that, in particular, in order to detect a trace amount of substance that is present in a biological sample in this system, a countermeasure against the background signal is very important and have arrived at the present invention through a measure for reducing the background signal level. Specifically, a reduction in nonspecific binding was significant after a cell homogenate extract and a biotin-binding protein were added to a biological sample. Alternatively, addition of a cell homogenate extract prepared from cells genetically engineered to bind a biotin-binding protein, instead of the biotin-binding protein, achieved substantially the same effect.

In addition, the present inventors have discovered that, in the system in which a fusion protein between a protein that specifically binds to a substance to be detected and a biotin-binding protein is immobilized on a carrier, a lower concentration of substance can be stably measured, with a reduced background signal level, by putting (blocking) the biotin-binding protein into contact with the carrier before the addition of a biological sample.

Based on the knowledge described above, the present invention provides a method of high-sensitive detection with reduced non-specific binding in a system in which a substance that specifically detects a substance to be detected is immobilized on a carrier through avidin-biotin binding.

The present invention includes the following nonlimiting embodiments.

### Embodiment 1

A method for detecting a substance in a biological sample, which comprises:

1) providing a carrier on which biotin is bound and providing a fusion protein between a protein that specifically binds to a substance to be detected and a biotin-binding protein;

2) binding the fusion protein to the carrier provided in step 1) through binding between biotin and the biotin-binding protein to produce a fusion protein-bound carrier;

3) mixing

(a) a biological sample, and

(b-i) a cell homogenate extract prepared from cells of the same species as the host cells used for expressing the fusion protein in step 1), and a biotin-binding protein, or

(b-ii) a cell homogenate extract prepared from cells of the same species as the host cells used for expressing the fusion protein in step 1), and genetically engineered to express a biotin-binding protein, and adding the mixture to the fusion protein-bound carrier produced in step 2); and

4) detecting a substance that has bound to the protein that specifically binds to a substance to be detected in the fusion protein.

### Embodiment 2

A method for detecting a substance in a biological sample, which comprises:

1) providing a carrier on which biotin is bound and providing a fusion protein between a protein that specifically binds to a substance to be detected and a biotin-binding protein;

2) binding the fusion protein to the carrier provided in step 1) through binding between biotin and the biotin-binding protein to produce a fusion protein-bound carrier;

3) putting a biotin-binding protein into contact with the fusion protein-bound carrier produced in step 2) to block the carrier;

4) after the blocking step in step 3), adding a biological sample to the fusion protein-bound carrier; and

5) detecting a substance to be detected that has bound to the protein that specifically binds to the substance in the fusion protein.

### Embodiment 3

A method for detecting a substance in a biological sample, which comprises:

1) providing a carrier on which biotin is bound and providing a fusion protein between a protein that specifically binds to a substance to be detected and a biotin-binding protein;

2) binding the fusion protein to the carrier provided in step 1) through binding between biotin and the biotin-binding protein to produce a fusion protein-bound carrier;

3) putting a biotin-binding protein into contact with the fusion protein-bound carrier produced in step 2) to block the carrier;

4) after the blocking step in step 3), mixing

(a) a biological sample, and

(b-i) a cell homogenate extract prepared from cells of the same species as the host cells used for expressing the fusion protein in step 1), and a biotin-binding protein, or

(b-ii) a cell homogenate extract prepared from cells of the same species as the host cells used for expressing the fusion protein in step 1), and genetically engineered to express a biotin-binding protein, and adding the mixture to the fusion protein-bound carrier; and

5) detecting a substance to be detected that has bound to the protein that specifically binds to the substance in the fusion protein.

### Embodiment 4

The method according to Embodiment 1 or 3, wherein step 3(b-i) in Embodiment 1 or step 4(b-i) in Embodiment 3 comprises adding a cell homogenate extract extracted from cells comprising any vector, as the cell homogenate extract.

### Embodiment 5

The method according to any one of Embodiments 1 to 4, wherein the biotin-binding protein is tamavidin or a variant thereof.

### Embodiment 6

The method according to any one of Embodiments 1 to 5, wherein the biological sample is selected from the group consisting of blood, serum, cerebrospinal fluid, saliva, sweat, urine, tear, lymph fluid, and mother's milk.

### Embodiment 7

A carrier for detecting a substance in a biological sample, wherein

a fusion protein between a protein that specifically binds to a substance to be detected and a biotin-binding protein is bound to a carrier by binding between biotin and the biotin-binding protein, wherein

the fusion protein is bound to the carrier by a method comprising:

1) providing a carrier on which biotin is bound and providing a fusion protein between a protein that specifically binds to a substance to be detected and a biotin-binding protein;

2) binding the fusion protein to the carrier provided in step 1) through binding between biotin and the biotin-binding protein to produce a fusion protein-bound carrier; and

3) putting a biotin-binding protein into contact with the fusion protein-bound carrier produced in step 2) to block the carrier.

### Embodiment 8

A kit for detecting a substance in a biological sample, which comprises:

A) a carrier on which a fusion protein between a protein that specifically binds to a substance to be detected and a biotin-binding protein is bound by binding between biotin and the biotin-binding protein; and

an agent for diluting a biological sample, comprising

B-i) a cell homogenate extract prepared from cells of the same species as the host cells used for expressing the fusion protein in step A), and a biotin-binding protein, or

B-ii) a cell homogenate extract prepared from cells of the same species as the host cells used for expressing the fusion protein in step A), and genetically engineered to express a biotin-binding protein; and/or

C) a blocking agent containing a biotin-binding protein.

### Advantageous Effects of Invention

The method of the present invention enables high-sensitivity and stable detection, with a reduced background signal level, of a substance to be detected in a biological sample. In particular, the method of the present invention enables detection of a trace amount of substance which is present in a biological sample and, usually, cannot be readily detected.

### BRIEF DESCRIPTION OF THE DRAWINGS

FIG. 1 includes schematic diagrams illustrating blocking by a biotin-binding protein. The open circles represent biotin, the closed ellipses represent a biotin-binding protein, and the open ellipses represent a protein that specifically binds to a substance to be detected: FIG. 1A: without blocking by a biotin-binding protein and with immobilization of a fusion protein; FIG. 1B: without blocking by a biotin-binding protein and without immobilization of a fusion protein; FIG. 1C: with blocking by a biotin-binding protein and with immobilization of a fusion protein; and FIG. 1D: with blocking by a biotin-binding protein and without immobilization of a fusion protein.

FIG. 2 illustrates detection by Western blotting of a fusion protein between SITH-1 and TM2 expressed in E. coli. E. coli containing a vector alone was used as a control.

FIG. 3 shows the effect of the addition of an E. coli homogenate extract to serum on non-specific binding and the blocking effect of tamavidin 2. FIG. 3-1 shows the result of blocking by BSA (only), that is, the result obtained by subtracting the measured value in a biotinylated plate (0.5% BSA Blocking) on which nothing is immobilized from the measured value in a SITH-1-TM2 fusion protein-immobilized plate (0.5% BSA Blocking). FIG. 3-2 shows the result of blocking by BSA and TM2, that is, the result obtained by subtracting the measured value in a biotinylated plate (50 μg/mL TM2/0.5% BSA Blocking) on which nothing is immobilized from the measurement value in a SITH-1-TM2 fusion protein-immobilized plate (50 μg/mL TM2/0.5% BSA Blocking).

FIG. 4 illustrates detection of a fusion protein between Harpin and TM2 expressed in E. coli in lane 2 by Western blotting. E. coli containing a vector alone was used (lane 1) as a control.

### EMBODIMENTS OF INVENTION

### I. Method of Detection of the Present Invention (Embodiment 1)

The method of detection (Embodiment 1) according to the present invention relates to a method for detecting a substance in a biological sample and comprises:

1) providing a carrier on which biotin is bound and providing a fusion protein between a protein that specifically binds to a substance to be detected and a biotin-binding protein;

2) binding the fusion protein to the carrier provided in step 1) through binding between biotin and the biotin-binding protein to produce a fusion protein-bound carrier;

3) mixing

(a) a biological sample, and

(b-i) a cell homogenate extract prepared from cells of the same species as the host cells used for expressing the fusion protein in step 1), and a biotin-binding protein, or

(b-ii) a cell homogenate extract prepared from cells of the same species as the host cells used for expressing the fusion protein in step 1), and genetically engineered to express a biotin-binding protein, and adding the mixture to the fusion protein-bound carrier produced in step 2); and

4) detecting a substance that has bound to the protein that specifically binds to a substance to be detected in the fusion protein.

Substance to be Detected in Biological Sample

The present invention relates to a method for detecting a substance in a biological sample.

Any biological sample that is expected to contain a substance as a detection target can be used in the present invention without limitation. Examples of the sample include cells and tissues collected from organisms and fragments thereof, for example, humors, more preferably, blood, serum, cerebrospinal fluid, saliva, sweat, urine, tear, lymph fluid, and mother's milk.

These humors may be used after dilution as needed. The dilution rate is, but not limited to, generally in the range of about 2 to about 10000 fold, preferably about 100 to 1000 fold. The diluent may be any buffer solution, which may contain any proper blocking agent. Preferred blocking agents have high inhibitory effect on nonspecific binding, and can be selected from blocking agents well-known to persons skilled in the art, such as BSA and casein. Note that in Embodiment 2 of the present invention, the blocking agent is a biotin-binding protein. This will be described below.

The substance to be detected in the present invention is any substance that is desired to be detected or measured in a biological sample, and preferred examples thereof include proteins such as antibodies and antigens and their fragments, peptides, nucleic acids, carbohydrates, and glycolipids.

The present invention enables measurement of a trace amount of substance that is present in a biological sample and cannot be readily detected or accurately determined quantitatively by conventional methods. For example, if the substance to be detected is an antibody exhibiting a low antibody titer in a serum (e.g., a low antibody titer not detectable at 1000-fold dilution, but detectable at 100-fold dilution with difficulty), a low dilution rate of the serum is required. As a result, non-specific binding derived from serum components inevitably increases. Thus, no known method enables detection or determination of quantity for the antibody. In contrast, the method of the present invention can readily detect and accurately determine quantity of the antibody.

Nonlimiting examples of the substance to be detected in the present invention include antibodies against a small protein encoded by the intermediate transcript of HHV-6 (SITH-1). Other examples include antibodies against other antigens of herpes viruses, antibodies against virus-related antigens derived from, for example, cytomegaloviruses, hepatitis viruses, HIVs, HTLVs, measles viruses, and influenza viruses, antibodies against bacterium-related antigens derived from, for example, Helicobacter pylori, and antibodies against fungi-related antigens.

SITH-1 Based on Description in PCT/JP2008/67300 and U.S. Provisional Application No. 61/102441

(1) SITH-1 Protein and Nucleic Acid

The structures and functions of the SITH-1 protein and a nucleic acid are disclosed in PCT/JP2008/67300, and the entity thereof is incorporated therein.

The SITH-1 is a factor involving latent infection with herpes viruses, and more particularly, a protein specifically expressed during latent infection with herpes viruses. The term “specifically expressed during latent infection with herpes viruses” therein refers to specific expression of genes or gene products derived from herpes viruses during latent infection (not productive infection) with herpes viruses in hosts infected with herpes viruses.

Examples of the SITH-1 protein and the nucleic acid include (a) a protein which has an amino acid sequence of SEQ ID NO: 1 and a nucleic acid encoding the protein.

The SITH-1 protein having the amino acid sequence of SEQ ID NO: 1, as described in Reference Example below, was isolated and identified as a protein that is specifically expressed during latent infection with human herpes viruses 6 (HHV-6). The SITH-1 protein is a protein having the amino acid sequence of SEQ ID NO: 1, composed of 159 amino acids, and having a molecular mass of about 17.5 kDa.

The SITH-1 protein is encoded by the nucleic acid of the SITH-1 gene. The cDNA of this SITH-1 gene, as shown in SEQ ID NO: 3, has a size of 1795 base pairs (about 1.79 kbp), the nucleotide sequence from the 954th to 956th being the initiation codon (Kozak ATG), while the nucleotide sequence from 1431st to 1433rd being the termination codon (TAA). Accordingly, the SITH-1 nucleic acid has a nucleotide sequence from 954th to 1430th as an open reading frame (ORF) in the nucleotide sequence of SEQ ID NO: 3, the ORF having a size of 477 base pairs (about 0.48 kbp). In the cDNA of the SITH-1, the nucleotide sequence representing the ORF region is shown in SEQ ID NO: 2. The nucleotide sequence of SEQ ID NO: 2 includes three bases of the stop codon.

The SITH-1 nucleic acid is always expressed in the cytoplasm of a cell latent-infected with HHV-6, but not in a productively infected cell. The nucleic acid encoding the SITH-1 protein is encoded by a DNA that is a complementary strand of the HHV-6 latent infection specific gene (H6LT), which has been reported to date, and its expression is enhanced in the intermediate stage of the latent infection with HHV-6. These facts demonstrate that the SITH-1 protein is a protein that is specifically expressed during latent infection with HHV-6.

The SITH-1 protein binds to a host protein, CAML (calcium-modulating cyclophilin ligand, Accesion #: U18242) to increase the calcium concentration in the glial cells. The CAML is a protein that is known to be abundantly present in the brain and lymphocytes in the host living organism and increase the intracellular calcium concentration. It is considered that an increase in intracellular calcium concentration due to expression of the SITH-1 protein probably leads to activation of overall signaling in the latent-infected cells, and thus contributes to efficient reactivation of HHV-6.

It is known that the glial cells in the brain are latent-infected with HHV-6. When HHV-6 during the latent infection or at the intermediate stage which is a latent infection state with high activity expresses the SITH-1, the calcium concentration seems to increase in the glial cells. It is believed that an increase in intracellular calcium concentration in the brain is wedded to psychiatric disorders such as mood disorders (Riken Annual Report 2003).

The SITH-1 protein has a function that maintains activity to bind to the host protein, CAML to increase the intracellular calcium concentration. Furthermore, expression of the SITH-1 protein in the glial cells, in which this protein seems to be most strongly expressed, in the brain can induce psychiatric disorders. Accordingly, the SITH-1 protein is believed to be expressed during the latent infection with herpes viruses or at the initial stage of reactivation of the herpes viruses to cause the host to have any psychiatric disorder.

(2) Antibody Against SITH-1

The antibody against the SITH-1 can be prepared as a polyclonal antibody or a monoclonal antibody from the SITH-1 protein, its variant, or their partial peptides as antigen by a known process. Examples of the known process are described in documents such as Harlow et al., “Antibodies: A laboratory manual (Cold Spring Harbor Laboratory, New York (1988))” and Iwasaki et al., “Monoclonal Antibody: Hybridoma and ELISA, Kodansha (1991)”. The resulting antibody can be used for detection and determination of the SITH-1 protein.

The term “antibody” refers to immunoglobulins (IgA, IgD, IgE, IgG, IgM, and Fab fragments, F(ab′)₂ fragments, and Fc fragments thereof). Examples of the antibody include, but not limited to, polyclonal antibodies, monoclonal antibodies, single-stranded antibodies, antiidiotype antibodies, and humanized antibodies.

The term “antibody recognizing the SITH-1 protein” includes complete molecules and antibody fragments specifically attachable to the SITH-1 protein (for example, Fab and F(ab′)₂ fragments). Fab, F(ab′)₂, and other fragments of the SITH-1 antibody can be used according to the method disclosed in the present specification or any known method. Such fragments can be typically produced by cleavage by proteolysis using an enzyme, e.g., papain (yielding a Fab fragment) or pepsin (yielding an F(ab′)₂ fragment).

It is believed that patients having mood disorders and individuals having potential mood disorders exhibit increased expression levels of the SITH-1 protein and thus increased SITH-1 antibody titers. In one embodiment of the present invention, detection of the SITH-1 antibody in a biological sample enables identification of patients having mood disorders and individuals having potential mood disorders.

Carrier on Which Fusion Protein Between Protein Specifically Binding to Substance to be Detected and Biotin-Binding Protein is Bound (Steps 1) and 2) of Example 1)

The method of detection according to the present invention uses a carrier on which a fusion protein between a protein that specifically binds to a substance to be detected and a biotin-binding protein is bound by binding between biotin and the biotin-binding protein.

The carrier of the present invention can be produced by a method including:

1) providing a carrier on which biotin is bound and providing a fusion protein between a protein that specifically binds to a substance to be detected and a biotin-binding protein; and

2) binding the fusion protein to the carrier provided in step 1) through binding between biotin and the biotin-binding protein to produce a fusion protein-bound carrier.

Biotin-Bound Carrier

“Biotin” is a generic name of D-[(+)-cis-hexahydro-2-oxo-1H-thieno-(3,4)-imidazole-4-valeric acid]. It is one of water-soluble vitamin categorized into a vitamin B group, and is also referred to as vitamin B₇, vitamin H, or coenzyme R. Biotin very strongly binds to avidin, one of the glycoproteins contained in egg white, so that its absorption is precluded. Thus, large dose of uncooked egg white may cause biotin deficiency disease in some cases.

The term “biotin” throughout the specification includes iminobiotin (Hofmann et al., (1980), Proc Natl Acad Sci USA, 77: 4666-4668), desthiobiotin (Hirsch et al., (2002), Anal Biochem, 308: 343-357), and biotin analogs such as biocytin and biotin sulfoxide, in addition to the biotin described above.

Systems using avidin (biotin-binding protein)/biotin complexes are widely used in the fields of tissue immunology, DNA analysis, and clinical assay. In the method for binding a protein to a carrier according to the present invention, a fusion protein composed of a biotin-binding protein and a desired protein is bound to a carrier by avidin-biotin binding. The method of the present invention ensures more efficient action of the protein, without impairing the function thereof, than conventional binding by avidin-biotin binding.

Examples of materials for the solid carrier include, but not limited to, cellulose, Teflon (registered trademark), nitrocellulose, agarose, dextran, chitosan, polystyrene, polyacrylamide, polyesters, polycarbonates, polyamides, polypropylene, nylons, polydivinylidene difluoride, latex, silica, glass, glass fiber, gold, platinum, silver, copper, iron, stainless steel, ferrite, silicon wafer, polyethylene, polyethyleneimine, poly(lactic acid), resin, polysaccharides, proteins such as albumin, carbon, and combination thereof. Preferred materials have a certain level of strength, a stable composition, and reduced non-specific binding.

Examples of the form of the solid carrier include, but not limited to, beads, magnetic beads, thin films, microtubes, filters, plates, microplates, carbon nanotubes, and sensor chips. Flat solid carriers such as thin films and plates may be provided with pits, grooves, or filter bottoms, as is known in the art.

In an embodiment of the invention, beads may have a spherical diameter in the range of about 25 nm to about 1 mm. In a preferred embodiment, the beads may have a diameter in the range of about 50 nm to about 10 μm. The size of the beads may be selected depending on the specific application. Since some bacterial spores have a size of an order of about 1 μm, preferred beads for capturing such spores have a diameter larger than 1 μm.

Without any limitation, for example, in the case that high sensitivity for detection is desired, beads as described above can preferably be used as the solid carrier in the view point that the beads provide high contacting frequency between the target substance to be detected and the substance that specifically binds to the target substance, and that cleaning operation thereof is easy.

An exemplary method of binding biotin to the carrier involves use of a biotinylation reagent. Examples of the biotinylation reagent include, but not limited to, EZ-Link (registered trademark) Sulfo-NHS-Biotin (the length of the linker: 13.5 angstroms, the reactive group: primary amine, hereinafter the same order), EZ-Link (registered trademark) Sulfo-NHS-LC-Biotin (22.4 angstroms, primary amine), EZ-Link (registered trademark) Sulfo-NHS-LCLC-Biotin (30.5 angstroms, primary amine), EZ-Link (registered trademark) PFP-Biotin (9.6 angstroms, amine), EZ-Link (registered trademark) Maleimide-PEO₂-Biotin (29.1 angstroms, thiol group), EZ-Link (registered trademark) Biotin-PEO₂ Amine (20.4 angstroms, carboxyl group), EZ-Link (registered trademark) Biotin-PEO₃-LC Amine (22.9 angstroms, carboxyl group), EZ-Link (registered trademark) Biotin-Hydrazide (15.7 angstroms, aldehyde group), EZ-Link (registered trademark) Biotin-LC-Hydrazide (24.7 angstroms, aldehyde group), and EZ-Link (registered trademark) NHS-Iminobiotin (13.5 angstroms, primary amine), which are available from PIERCE.

Using these biotinylation reagent, biotin can be bound to a desired carrier such a microplate, microbeads, or a sensor chip by any known process. For example, various carriers having functional groups, such as amino, carboxyl, thiol, tosyl, epoxy, and maleimide groups, and activated ester (for example, magnetic beads, Sepharose beads, agarose beads, latex beads, and microtiter plates) can be used. For example, in the case of the use of a biotinylation reagent containing NHS ester, the reagent may be dissolved in an organic solvent such as dimethyl sulfoxide (DMSO) or phosphate buffer of pH 7 to 9, and then may be added to an immobilization carrier having amino groups to bind biotin thereto. In the case of the use of a biotinylation reagent containing amino groups, the carboxyl groups on the immobilization carrier may be converted to activated ester using carbodiimide such as 1-ethyl-3-(3-dimethylaminopropyl) carbodiimide hydrochloride (EDC), followed by addition of a biotinylation reagent dissolved in buffer solution (pH: about 5) to bind biotin to the carrier. The biotinylated immobilization carrier is preferably blocked with BSA after inactivation of unreacted functional groups.

Commercially available biotinylated carriers can also be used. Examples of the biotinylated microplates include, but not limited to, Reacti-Bind™ Biotin Coated Polystyrene Plates (PIERCE). Examples of the biotinylated microbeads include, but not limited to, magnetic beads, such as BioMag Biotin (available from Polysciences), magnetic nanobeads, such as nanomag (registered trademark)-D biotin and nanomag (registered trademark)-silica biotin available from Corefront, polystyrene microbeads, such as Beadlyte (registered trademark) Biotin Beads (available from Upstate), agarose, such as Biotin Agarose and 2-iminobiotin-Agarose available from Sigma, and highly cross-linked agarose, such as Biotin-Sepharose (available from Biosearch Technologies, Inc.).

The length of the linker binding the carrier to biotin is preferably at least 5 angstroms, more preferably at least 13.5 angstroms, more preferably at least 22.4 angstroms, and most preferably at least 30.5 angstroms.

Protein Specifically Binds to Substance to be Detected

The protein that specifically binds to a substance to be detected (hereinafter may be referred to as “specific protein”) is not particularly limited In one embodiment of the present invention, for example, one of the antigen and antibody, ligand such as hormone and receptor, lectin and saccharide, or complementary bindings of nucleic acid in a sample to be tested is selectively analyzed by means of the ability to form a specific complex with the other, but the specific protein is not limited thereto.

More specifically, examples of the protein include, but not limited to, antibodies, antigenic proteins, lectins, peptides, protein A, protein protein L, receptors, and enzymatic proteins. Examples of the antibody include antibody fragments containing antigen-binding sites, such as scFv and Fab, in addition to IgG. Examples of the antigenic protein include proteins derived from viruses such as hepatitis B and C viruses, HIVs, influenza viruses, and herpes viruses; proteins derived from bacteria such as Helicobacter pylori; tumor markers such as CEA and PSA; and sex hormones. The lectin is a saccharide-binding protein, and examples thereof include monosaccharide specific lectins such as mannose specific lectin, GalNAc specific lectin, GlcNAc specific lectin, fucose specific lectin, and sialic acid specific lectin, and oligosaccharide specific lectins. Furthermore, the examples include DNA/RNA binding proteins. Examples of the peptide include those composed of 2 to 100 amino acids, preferably 4 to 50 amino acids, and more preferably 6 to 30 amino acids.

Furthermore, in the present invention, examples of the protein that specifically binds to a substance to be detected include, but not limited to, SITH-1 proteins.

Biotin-Binding Protein

The present invention includes a method of immobilizing a protein that specifically binds to a substance to be detected to a carrier by binding between biotin and a biotin-binding protein. In the present invention, “biotin and a biotin-binding protein” may be referred to as “avidin-biotin binding” in some cases.

Any protein that strongly binds to biotin can be preferably used as the biotin-binding protein, and examples thereof include avidin, streptavidin, neutravidin, AVR protein (Biochem. J., (2002), 363: 609-617), bradavidin (J. Biol. Chem., (2005), 280: 13250-13255), rhizavidin (Biochem. J., (2007), 405: 397-405), tamavidin (WO2002/072817), and variants thereof. The dissociation constant (KD) with biotin is preferably 10⁻⁶ M or less, more preferably 10⁻⁸ M or less, more preferably 10⁻¹⁰ M or less. Note that the biotin-binding protein that is added to a substance to be tested and the biotin-binding protein that is used for blocking the carrier will be described below.

Particularly preferred biotin-binding proteins are tamavidin and variants thereof, which can be highly expressed in E. coli. Tamavidin is a biotin-binding protein discovered in an edible mushroom, Pleurotus cornucopiae (WO2002/072817, Takakura et al., (2009), FEBS J, 276: 1383-1397). An example of the variants of tamavidin is tamavidin exhibiting high binding capability and low non-specific binding characteristics (PCT/JP2009/64302).

The term “tamavidin” in the present invention refers to tamavidin 1, tamavidin 2, or a variant thereof. Specifically, tamavidin of the present invention may be typically a protein having the amino acid sequence of SEQ ID NO: 5 or SEQ ID NO: 7, or a protein encoded by a nucleic acid having the nucleotide sequence of SEQ ID NO: 4 or SEQ ID NO: 6. Alternatively, tamavidin of the present invention may be a protein that is a variant of a protein having the amino acid sequence of SEQ ID NO: 5 or SEQ ID NO: 7 or a protein encoded by a nucleic acid having the nucleotide sequence of SEQ ID NO: 4 or SEQ ID NO: 6 and having biotin binding capability similar to that of tamavidin 1 or 2 or high binding capability and low non-specific binding characteristics. Throughout the specification, tamavidin 1, tamavidin 2, and variants thereof may be collectively referred to as tamavidin.

The variant of tamavidin 1 or 2 may be a protein having an amino acid sequence comprising one or more deletion, substitution, insertion, and/or addition of one or more amino acids in the amino acid sequence of SEQ ID NO: 5 or 7 and having biotin binding capability similar to that of tamavidin 1 or 2. The substitution may be conservative substitution. The conservative substitution refers to replacement of a specific amino acid residue with any residue having similar physicochemical features. Nonlimiting examples of the conservative substitution include substitutions between amino acid residues containing aliphatic groups, such as mutual substitution between Ile, Val, Leu, and Ala; and substitutions between polar residues, such as mutual substitution between Lys and Arg, between Glu and Asp, and between Gln and Asn.

The variant by deletion, substitution, insertion, and/or addition of an amino acid or amino acids can be produced by a known technique such as site-specific mutagenesis (e.g., see Nucleic Acid Research, Vol. 10, No. 20, pp. 6487-6500, 1982, which is incorporated herein in its entirety) to a DNA encoding a wild-type protein. Throughout the specification, the term “one or more amino acids” herein refers to an amino acid or amino acids that can be deleted, substituted, inserted, and/or added by preferably site-specific mutagenesis. In addition, the term “one or more amino acids” herein may refer to one or more amino acids. The “one or several amino acids” refers to, but not limited to, 50 or less, preferably 40 or less, 30 or less, 20 or less, 10 or less, 8 or less, 5 or less, or 3 or less amino acids. The variant of tamavidin 1 or 2 may also be a protein having an amino acid sequence sharing an identity of 60% or more, preferably 65% or more, 70% or more, 75% or more, 80% or more, 85% or more, 90% or more, 95% or more, 96% or more, 97% or more, 98% or more, or 99% or more, more preferably 99.3% or more with that of SEQ ID NO: 5 or SEQ ID NO: 7 and having biotin binding capability similar to that of tamavidin 1 or 2 or high binding capability and low non-specific binding characteristics.

The percent identity between two amino acid sequences may be determined by visual inspection and mathematical calculation. Alternatively, the percent identity between two protein sequences may be determined through comparison of sequence information using a GAP computer program available from the University of Wisconsin Genetics Computer Group (UWGCG) based on the algorithm by Needleman, S. B. and Wunsch, C. D. (J. Mol. Biol., 48: 443-453, 1970). Preferred default parameters of the GAP program include: (1) scoring matrix: blosum62 described in Henikoff, S. and Henikoff, J. G., (Proc. Natl. Acad. Sci. USA, 89: 10915-10919, 1992); (2) 12 gap weights; (3) 4 gap length weights; and (4) no penalty for terminal gaps.

Any other program used by persons skilled in the art may also be used for comparison of the sequences. The percent identity can be determined by, for example, comparison with the sequence information using a BLAST program described in Altschul et. al., (Nucl. Acids. Res., 25, pp. 3389-3402, 1997). This program is available from the websites of National Center for Biotechnology Information (NCBI) or DNA Data Bank of Japan (DDBJ) on the Internet. The conditions (parameters) for identity search by the BLAST program is described in detail on these sites. Although these parameters can be partly modified if necessary, search is generally carried out using the default values. Alternatively, the percent identity between two amino acid sequences may be determined using a program such as genetic information processing software GENETYX Ver. 7 (available from GENETYX CORPORATION) or FASTA algorithm, wherein search may be carried out using the default values.

The percent identity between two nucleotide sequences can be determined by visual inspection and mathematical calculation. Preferably, such comparison is carried out through comparison of sequence information using a computer program. A particularly preferred computer program is a version 10.0 program “GAP”, Wisconsin package of Genetics Computer Group (GCG, Madison, Wis.) (Devereux, et al., 1984, Nucl. Acids Res., 12: 387). The use of the “GAP” program enables comparison between two amino acid sequences and comparison between a nucleotide sequence and an amino acid sequence, in addition to comparison of two nucleotide sequences.

The “biotin-binding protein” constituting a fusion protein to be immobilized to a carrier is used for preparing a fusion protein-bound carrier by binding the fusion protein to the carrier through binding between biotin and the biotin-binding protein. Accordingly, it is preferred that the biotin binding activity of a variant of tamavidin 1 or 2 be not significantly decreased compared to that in the case of forming the fusion proteins using these wild-types.

Accordingly, nonlimitingly, in the variant of tamavidin 1, preferably, N14, S18, Y34, S36, S78, W82, W98, W110, and D118 in the amino acid sequence of SEQ ID NO: 5 are not modified. Note that the notation, for example, Y34 indicates the 34th tyrosine residue of the amino acid sequence of SEQ ID NO: 5. Alternatively, in the modification of these amino acid, the amino acid is preferably replaced with one having a similar property or structure. For example, asparagine (N14) is replaced with glutamine (Q) or aspartic acid (D), preferably aspartic acid; serine (S18, S36, or S78) is replaced with threonine (T) or tyrosine (Y), preferably threonine; tyrosine (Y34) is replaced with serine (S), threonine (T), or phenylalanine (F), preferably phenylalanine; tryptophan (W82, W98, or W110) is replaced with phenylalanine (F); and aspartic acid (D118) is replaced with glutamic acid (E) or asparagine (N), preferably asparagine.

In the variant of tamavidin 2, preferably, four tryptophan residues (W69, W80, W96, and W108) in the amino acid sequence of SEQ ID NO: 7 are not modified. Alternatively, when these amino acid residues are modified, the amino acid is preferably replaced with one having a similar property or structure, for example, phenylalanine (F). In addition, it is desirable that amino acid residues (N14, S18, Y34, S36, S76, T78, and D116) that probably interact directly with biotin are also not modified. Alternatively, in the modification of these amino acid residues, the amino acid is preferably replaced with one having a similar property or structure in order to maintain the binding with biotin. For example, asparagine (N14) is replaced with glutamine (Q) or aspartic acid (D), preferably aspartic acid; aspartic acid (D40) is replaced with asparagine (N); serine (S18, S36, or S76) is replaced with threonine (T) or tyrosine (Y), preferably threonine; tyrosine (Y34) is replaced with serine (S), threonine (T), or phenylalanine (F), preferably phenylalanine; threonine (T78) is replaced with serine (S) or tyrosine (Y), preferably serine; and aspartic acid (D116) is replaced with glutamic acid (E) or asparagine (N), preferably asparagine.

Preferred variants of tamavidin in the present invention include the following variants (PCT/JP2009/64302).

The tamavidin variant is a modified biotin-binding protein that has the amino acid sequence of SEQ ID NO: 7 or an amino acid sequence comprising one to several amino acid mutations in this sequence or having an identity of at least 80% with this sequence and that shows biotin binding activity, wherein one or more residues selected from the group consisting of:

1) the arginine residue at the 104th site of SEQ ID NO: 7;

2) the lysine residue at the 141st site of SEQ ID NO: 7;

3) the lysine residue at the 26th site of SEQ ID NO: 7; and

4) the lysine residue at the 73rd site of SEQ ID NO: 7;

are replaced with acidic or neutral amino acid residues.

More preferably, the modified biotin-binding protein is selected from the group consisting of:

a modified biotin-binding protein (R104E-K141E) in which the arginine residue at the 104th site is replaced with a glutamic acid residue, and the lysine residue at the 141st site is replaced with a glutamic acid residue, in SEQ ID NO: 7;

a modified biotin-binding protein (D40N-R104E) in which the aspartic acid residue at the 40th site is replaced with a asparagine residue, and the arginine residue at the 104th site is replaced with a glutamic acid residue, in SEQ ID NO: 7;

a modified biotin-binding protein (D40N-K141E) in which the aspartic acid residue at the 40th site is replaced with a asparagine residue, and the lysine residue at the 141st site is replaced with a glutamic acid residue, in SEQ ID NO: 7; and

a modified biotin-binding protein (D40N-R104E-K141E) in which the aspartic acid residue at the 40th site is replaced with a asparagine residue, the arginine residue at the 104th site is replaced with a glutamic acid residue, and the lysine residue at the 141st site is replaced with a glutamic acid residue, in SEQ ID NO: 7.

Fusion Protein Between Protein Specifically Binding to Substance to be Detected and Biotin-Binding Protein

In the present invention, a fusion protein between a protein that specifically binds to a substance to be detected and a biotin-binding protein (hereinafter may be referred to as “biotin-binding protein fusion protein” or “fusion protein”) is immobilized on a carrier.

The method for preparing the biotin-binding protein fusion protein is not particularly limited. For example, the fusion protein may be expressed by a known genetic engineering technique. For example, the fusion protein can be obtained by expressing a gene encoding a fusion protein between a biotin-binding protein and a desired protein using an expression system such as E. coli. Note that tamavidin and variants thereof are preferred to achieve a high expression in E. coli.

In the biotin-binding protein fusion protein, a biotin-binding protein and a desired protein may be directly bound to each other or may be bound via a linker, but are preferably bound via an amino acid linker. The length of the linker may be at least one amino acid, but is preferably at least five amino acids and more preferably at least six amino acids. In order to enhance the binding strength between biotin immobilized on a carrier and tamavidin, the length of the linker is preferably at least ten amino acids, more preferably at least 12 amino acids, at least 15 amino acids, at least 18 amino acids, and most preferably at least 25 amino acids. Probably, such linkers also increase the activity of the tamavidin fusion protein. The amino acids constituting the linker are not particularly limited, and the linker is preferably made of repetition of neutral amino acids such as glycine, serine, or alanine. Nonlimiting examples thereof include GGGGS, GGSGG, GASAG, GSGAA, GSGSA, GGGGSG, GGGSGGS, GGSGGGGS, AAAAGSGAA, GGGGSGGGGSGGGGS, and GGGGSGGGGSGGGGSGGGGSGGGGS (SEQ ID NOs: 8 to 18).

The biotin-binding protein may be bound to either the N-terminal or the C-terminal of a desired protein. In the expression of a desired protein, for example, if the periplasmic space is more suitable than the cytoplasm of E. coli, a leader sequence for targeting the periplasm may be used. Examples of the leader sequence include, but not limited to, Pe1B (Lei et al., (1987), J Bacteriol, 169: 4379-4383) and OmpA (Gentry-Weeks et al., (1992), J Bacteriol, 174: 7729-7742).

In the case of a biotin-binding protein fusion protein obtained from a soluble fraction, the soluble fraction, without purifying the crude protein extract, may be put into direct contact with a biotinylated carrier to bind the fusion protein to the biotinylated carrier. Through sufficiently washing the carrier, the purification and the immobilization to the carrier of the fusion protein can be simultaneously achieved. Alternatively, the fusion protein may be bound to the biotinylated carrier after purification using a column to which a biotin analog such as iminobiotin Hofmann et al., (1980), Proc Natl Acad Sci USA, 77: 4666-4668) is bound.

Alternatively, a tag for purification may be further added to the N-terminal or the C-terminal of the biotin-binding protein fusion protein. Examples of the tag include, but not limited to, a c-myc epitope tag (Munro and Pelham, (1986), Cell, 46: 291-300), a histidine tag (Hochuli et al., (1988), Bio/Technol, 6: 1321-1325; Smith et al., (1988), J Biol Chem, 263: 7211-7215), a Halo tag (Los and Wood, (2007), Methods Mol Biol, 356: 195-208), a Flag tag (Einhauer and Jungbauer, (2001), J Biochem Biophys Methods, 49: 455-465), and combinations thereof.

In the case of a biotin-binding protein fusion protein obtained from an insoluble fraction, a known process employed, for example, the protein is once solubilized using a chaotropic salt such as urea or guanidine hydrochloride, and then refolding of the protein is enhanced with gradually removing the chaotropic salt by, for example, dialysis (Sano and Cantor, (1991), Bio/Technology, 9: 1378-1381; Sano et al., (1992), Proc Natl Acad Sci USA, 89: 1534-1538).

In the case of a desired protein expressed in an insoluble fraction in E. coli, for example, a maltose-binding protein (Bach et al., (2001), J Mol Biol, 312: 79-93), thioredoxin (Jurado et al., (2006), J Mol Biol, 357: 49-61), glutathione S-transferase (Tudyka and Skerra, (1997), Protein Sci, 6: 2180-2187), or a chaperon such as one described in Ideno et al., (2004), Appl Microbiol Biotechnol, 64: 99-105 may be coexpressed, or a three-component fusion protein composed of the fusion protein and a chaperon may be produced. The maltose-binding protein, the thioredoxin, and the glutathione S-transferase can also be used as tags for purification.

The fusion protein may be expressed by any other known expression system, such as insect cells, plant cells, mammalian cells, yeast cells, Bacillus subtilis cells, or a cell-free expression system. In particular, when the protein to be fused is expressed in plant cells (for example, plant lectin), it is preferred that the fusion protein is also expressed in a plant cell expression system. A suitable expression system will be apparent to those skilled in the art in consideration of properties of the protein to be fused.

Binding of Fusion Protein to Carrier

In the present invention, a protein can be bound to a carrier through avidin-biotin binding by providing a biotin-bound carrier and a biotin-binding protein fusion protein and putting them into contact with each other.

A crude cell homogenate extract containing a biotin-binding protein fusion protein is prepared in a total protein content of, but is not limited to, 0.1 mg/mL to 5 mg/mL, preferably 0.2 mg/mL to 2 mg/mL. This extract is put into contact with a carrier to which biotin is bound at 10° C. to 40° C., preferably 20° C. to 30° C., for 5 min to 2 hr, preferably 30 min to 1 hr. Alternatively, a purified biotin-binding protein fusion protein in a concentration of 0.1 μg/mL to 5 μg/mL may be put into contact with a carrier to which biotin is bound.

### Addition of Biological Sample to Carrier (Step 3 of Embodiment 1)

The method of Embodiment 1 of the present invention includes, after the providing of a fusion protein-bound carrier, the following step 3):

mixing

(a) a biological sample, and

(b-i) a cell homogenate extract prepared from cells of the same species as the host cells used for expressing the fusion protein in step 1), and a biotin-binding protein, or

(b-ii) a cell homogenate extract prepared from cells of the same species as the host cells used for expressing the fusion protein in step 1), and genetically engineered to express a biotin-binding protein, and adding the mixture to the fusion protein-bound carrier produced in step 2).

In a method of detection using a carrier, generally known approaches for reducing non-specific binding which causes background signals are, for example, addition of an extract of a bacterium component to a reagent for detection (JP No. S59-99257 A (1984)); addition of a culture component of host cells to a sample, where a vector of the same species as that used in production of a recombinant protein capable of specifically binding to a substance to be detected and containing no gene encoding the protein (JP No. H8-43392 A (1996)); heat treatment of an aqueous extract from cells of the same species as that producing a recombinant protein capable of specifically binding to a substance to be detected and not containing this protein, and addition of its water-soluble fraction to a sample (JP No. 2004-301646 A).

The present inventors had attempted to apply the above-mentioned methods to a system of fusion protein, but sufficient effect has was not obtained. However, the present inventors have diligently studied and, as a result, have arrived at a method of obtaining a noticeable effect. Specifically, the present inventors have discovered that it is preferable to put a substance to be detected into contact with a carrier in the presence of both a cell homogenate extract and a biotin-binding protein.

In one embodiment, a cell homogenate extract prepared from cells of the same species as the host cells used for expressing the fusion protein, biotin-binding protein, and a biological sample are mixed, and the mixture may be added to a carrier. When a biotin-binding protein and a cell homogenate extract are added to a sample to be tested, either one may be added first, or the both may be simultaneously added. Alternatively, after mixing the biotin-binding protein and the cell homogenate extract, the sample to be tested may be added thereto.

In another embodiment, a cell homogenate extract prepared from cells of the same species as the host cells used for expressing the fusion protein and genetically engineered to express a biotin-binding protein and a sample to be tested are mixed, and the mixture may be added to a carrier. Specifically, a recombinant protein is expressed in cells by integrating the biotin-binding protein into an expression vector, and the homogenate extract from the cells may be used.

In the case of a serum sample to be tested, the serum is usually used after dilution in a cell homogenate extract into 10 to 10000-fold, preferably 100 to 1000-fold, more preferably 100 to 500-fold.

Cells from which cell homogenate extracts are derived, such as E. coli cells, yeast cells, mammalian cells, insect cells, and plant cells, can be used without limitation. Preferred cells are the same species as that of host cells used to express a fusion protein. For example, in the case of preparation of a fusion protein using E. coli, it is preferred that the cell homogenate extract be also prepared using E. coli. In the case of expression of a fusion protein in a cell-free system, the cell homogenate extract used can be used as it is or as a suspension in a desired buffer solution.

More preferably, the cells are derived from different species from the organism from which the biological sample is derived. For example, for a biological sample derived from human being, a cell homogenate extract is preferably prepared from cells derived from an organism other than human beings. However, for example, for a fusion protein expressed in mammalian cells, a cell line obtained from specific cancer cells is generally used. In such a case, the content of the human cell line and the content of (for example) human cells from which the biological sample are derived are substantially highly different from each other, even if the cell homogenate extract from a cell line derived from human being is used; hence, the effect of the present invention can be obtained.

The cell used for preparation of the cell homogenate extract may contain any vector, preferably an empty vector. The empty vector is of the same species as that of the vector used in expression of a protein that specifically binds to substance to be detected or a fusion protein thereof and does not contain genes that encode these proteins. The empty vector may further contain any nucleic acid. Alternatively, the empty vector may be any known vector that is different from the vector used in expression of a protein that specifically binds to substance to be detected or a fusion protein thereof.

Any cell homogenate extract derived from cells can be used without limitation. For example, a protein component, a carbohydrate component, or a lipid component of cells, or a mixture thereof may be used. Preferably, a soluble extract of cells can be used.

The cell homogenate extract can be prepared by a variety of processes without limitation. Usually, the cell homogenate extract can be prepared as a soluble components by homogenizing or solubilizing cells cultured in a proper culture medium through a physical means such as, ultrasonic application, a chemical means using a surfactant, or an enzymatic treatment, and performing centrifugation or filtration. In order to prolong the storage life, preferably the clear liquid prepared by centrifugation or filtration is mixed with, for example, a protease inhibitor, or is heated, for example, in an autoclave to suppress or deactivate various enzymes derived from cells. The concentration of the cell homogenate extract can be varied depending on the extent of the occurring non-specific reaction and can be set in a range sufficient to absorb the non-specific reaction.

As a nonlimiting specific example of the method for preparing a cell homogenate extract, in the case of E. coli cells, E. coli cells (which may contain a vector optionally containing a gene encoding a biotin-binding protein) are inoculated into an LB culture medium containing an antibiotic; shake culture is performed at 25° C. to 37° C. until the absorbance at OD 600 reaches 0.25 to 1, preferably 0.4 to 0.6; 0.1 mM to 5 mM, preferably 0.5 mM to 1 mM IPTG is added thereto; and then shake culture is performed at 25° C. to 37° C. for 2 to 24 hr, preferably 4 to 16 hr. The bacterial cells are recovered by centrifugation from the culture solution, are suspended in a desired buffer solution, and are homogenized. After centrifugation of the homogenized solution, the supernatant is collected as a crude E. coli extract.

The biological sample and cell homogenate extract can be added to a carrier by any procedure. The biological sample, however, must come into contact with the cell homogenate extract before the contact with the carrier. In other words, as long as the biological sample is put into sufficient contact with the cell homogenate extract, the component derived from the cell homogenate extract is not necessarily added to the carrier finally together with the biological sample. For example, a carrier to which the cell homogenate extract component is bound is prepared, and a treated biological sample may be added thereto. Specific examples include an embodiment in which a biological sample is passed through a cell homogenate extract component column.

When a biological sample is mixed with a crude cell homogenate extract, the sample is reacted with, but not limited to, a crude cell homogenate extract that has been prepared with a desired buffer (which may contain, for example, BSA, casein, or a commercially available blocking agent) into a total protein concentration of 0.05 to 5 mg/mL, preferably 0.5 to 5 mg/mL, at 10° C. to 30° C., preferably, 20° C. to 30° C. for 30 min to 4 hr, preferably 1 to 2 hr. For a biological sample of serum, the serum is preferably diluted with, but not limited to, the crude cell homogenate extract to 100 to 1000-fold.

Addition of Biotin-Binding Protein to Sample to be Tested

The present invention is characterized in that a mixture of a biological sample, a cell homogenate extract prepared from cells of the same species as the host cells used for expressing a fusion protein, and a biotin-binding protein is added to a carrier (step b-i), or a mixture of a sample to be tested and a cell homogenate extract prepared from cells of the same species as the host cells used for expressing a fusion protein and genetically engineered to express a biotin-binding protein is added to a carrier (step b-ii).

In the method of the present invention, the background signal level can be finally suppressed by adding a biotin-binding protein to a sample to be tested.

Such a biotin-binding protein may be the same as or different from the biotin-binding protein constituting a fusion protein. Either the wild type or a variant may be used. The biotin-binding activity of the variant may be equivalent, higher, or lower compared to that of the wild type.

In an embodiment of the addition, powder of a biotin-binding protein (which may be a naturally occurring protein or may be expressed by genetic engineering) may be added to the sample directly or after its dissolution in a proper liquid. For example, in another embodiment, a mixture of a sample and a cell homogenate extract may be treated with a carrier to which the biotin-binding protein is immobilized (for example, the mixture is passed through a column) (step b-i), instead of direct addition of the biotin-binding protein to the sample.

In the case of addition of the biotin-binding protein to a crude cell homogenate extract, the final concentration of the added biotin-binding protein is, but not limited to, 1 to 500 μg/mL, preferably 10 to 100 μg/mL. The concentration of the biotin-binding protein that is genetically engineered to be expressed in the cells may also substantially be the same as above, but any other concentration is acceptable.

Alternatively, a gene encoding a biotin-binding protein is introduced into host cells and is expressed, and a cell extract containing a biotin-binding protein obtained by homogenizing the host cells may be used (step b-ii). In this case, the biotin-binding protein may be expressed in a desired host by a method well known to persons skilled in the art. In the case where a fusion protein between a protein that specifically binds to a substance to be detected and a biotin-binding protein is expressed by genetic engineering, the cell homogenate extract is preferably derived from the same species as that of the host.

In the case of the host being E. coli, a gene encoding the biotin-binding protein is incorporated into an expression vector and is introduced into E. coli, and E. coli is cultivated while expression of protein is induced. Conditions for induction, such as an expression vector and host E. coli strains, culture medium component, IPTG concentration, and cultivation temperature can be appropriately determined.

Method of Detection of Substance to be Detected (Step 4 of Embodiment 1)

The method of detection according to the present invention detects a substance bound to the protein that specifically binds to a substance to be detected in a fusion protein.

Persons skilled in the art can appropriately select the method for detecting substance to be detected based on the properties of a desired protein. Preferred examples of such a method include immunoassays such as enzyme-linked immunosorbent assay (ELISA, including sandwich ELISA)) and radioimmunoassay (RIA); and other assays such as nucleic acid hybridization assay and surface plasmon resonance. After a sample to be tested is reacted with a substance that specifically binds and interacts with a substance to be detected and is immobilized by avidin-biotin binding, and the substance is detected.

In the immunoassay, for example, of a substance to be detected of an antibody, an antigen is immobilized and is reacted with the antibody present in a sample to be tested, and the reaction product is detected by any method well known to persons skilled in the art. For example, in the case where the sample to be tested is one derived from human being, the human antibody bound to the antigen is detected using an anti-human antibody. In this procedure, the anti-human antibody is labeled with a phosphor, enzyme, or radioisotope, and the fluorescence intensity, enzyme activity, or radiation dose is finally measured to indirectly measure or determine the amount of the antibody. When the substance to be determined is an antigen, an antibody against one site (epitope) of the antigen is immobilized and is reacted with the antigen present in a sample to be tested, and an antibody against another epitope of the antigen is further reacted with the antigen. In this procedure, this secondary antibody against the other epitope is labeled as described above to indirectly measure the amount of the antigen. Alternatively, in the nucleic acid hybridization assay, a nucleic acid composed of several tens of nucleotides to several hundreds or several thousands nucleotides and having a sequence region complementary to a nucleic acid to be determined is immobilized by avidin-biotin binding. This is reacted with a sample to be tested containing a nucleic acid labeled with a phosphor or radioisotope, and the amount of the phosphor or isotope is measured.

Any labeling well known to persons skilled in the art may be employed. Alternatively, commercially available phosphor- or enzyme-labeled anti-human antibodies may be used. Examples of the phosphor labeling include labeling using, for example, fluorescein or rhodamine, and labeling using a fluorescent protein such as a green fluorescent protein (GFP). Enzymes used for enzyme labeling are, but not limited to, peroxidase, alkaline phosphatase, luciferase, and glucose oxidase. Substrates for measurement using these enzymes are commercially available. For example, TBA and substrates for chemiluminescence can be used for peroxidase. Examples of the radioisotope include iodine (¹²⁵I and ¹²¹I), carbon (¹⁴C), sulfur (³⁵S), and tritium (³H), and phosphorus (³²P) for nucleic acids.

The amount of an antigen or an antibody present in the biological sample (specimen) can be readily calculated by comparison with the amount present in a standard preparation (e.g., a standard sample of a healthy subject or a typical patient in the case of clinical samples) using a linear regression computer algorithm. Such assay for detecting an antigen or an antibody, for example, ELISA is disclosed in Iacobelli et al., Breast Cancer Research and Treatment, 11: 19-30 (1988).

For example, in the case that the amount of the substance to be detected, e.g., antibody, in a biological sample is small (the case of a low antibody titer) or that the number of non-specific binds due to a biological sample, such as serum, per se is large, the effect of background signals due to non-specific binding would be noticeable. Accordingly, the substance to be detected can be more precisely determined by appropriately subtracting the background signals from the measured value. Those skilled in the art can appropriately determine the background signal to be subtracted depending on each experimental system.

For example, an example of such an embodiment is a “Human/Monkey anti-type I and type II Collagen IgG Antibody assay kit” (manufactured by Chondrex), which detects an antibody against collagen present in serum. In this kit, the background value (measured value of the secondary antibody only without addition of serum) is subtracted from the measured value of a sample.

In the case where the number of non-specific binds due to a biological sample per se is large, such as in case of serum, in order to subtract the background due to the non-specific reaction, as described in Example 2, the following embodiment is effective. The measured value in the section where an SITH-1 antigen (protein that specifically binds to the substance to be detected) is not immobilized (however, in the section, blocking with BSA or the like has been operated, and the serum (biological sample) containing the anti-SITH-1 antibody (the substance to be detected in the biological sample) has been added, as in the section where the SITH-1 antigen is immobilized) is subtracted from the measured value at the carrier (plate) on which a fusion protein between the SITH-1 antigen and tamavidin is immobilized. Alternatively, as described in Example 2 or 3, the amount may be calculated by subtracting the measured value in a carrier where only tamavidin is immobilized (bound) is subtracted from the measured value in a carrier (plate) where a fusion protein between an SITH-1 antigen and tamavidin is immobilized.

In another example of the embodiment for subtracting non-specific reaction specific to sample per se, as the above-mentioned “Human/Monkey anti-type I and type H Collagen IgG Antibody assay kit”, the measured value at a well on which collagen is not immobilized may be subtracted from the measured value of a serum collagen antibody at a well of a microtiter plate on which collagen is immobilized. Furthermore, a kit of a similar embodiment is “Virus antibody ETA “Seiken”Herpes IgM” (manufactured by Denka Seiken) that is used for detecting an anti-simplex herpes virus IgM antibody in serum or plasma.

Preferably, the amount can be more precisely determined by subtracting the measured value in the section where an arbitrary protein of which antibody is not possessed by an organism (e.g., human being in the case of SITH-1) from which the sample is derived is immobilized (a non-limiting example of the immobilized protein is GFP, when the organism is a mammalian). The method of immobilization is not particularly limited, and, preferably, a fusion protein with a biotin-binding protein is produced and is immobilized on a biotinylated carrier by avidin-biotin binding.

Persons skilled in the art can appropriately design and select the method of calculation as in above based on the properties of a biological sample and the characteristics of an antibody used.

The method of detection according to the present invention can specifically detect an antibody having a low antibody titer in, preferably, serum.

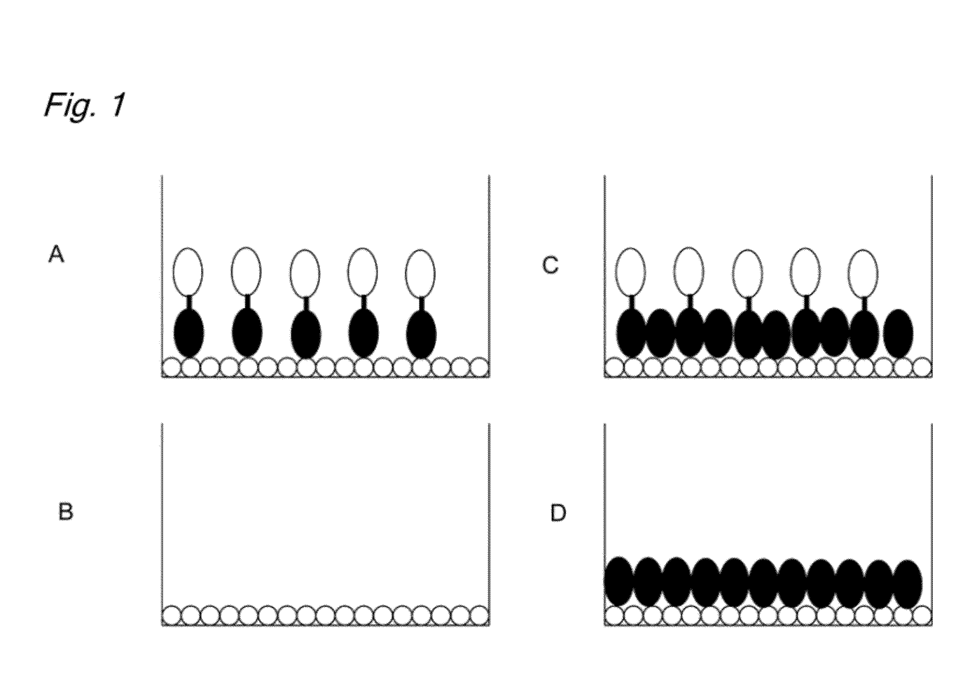

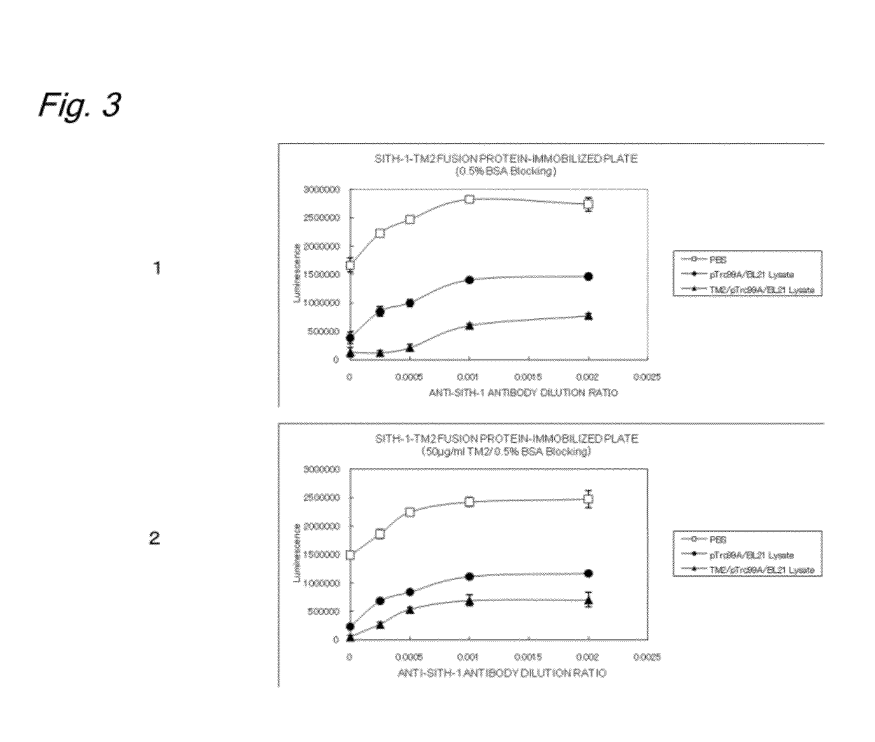

The effect of Embodiment 1 of the present invention can be apparent by, for example, comparing the results of TM2/pTrc99A/BL21 cell homogenate extract (closed triangle) in FIG. 3-1 with the results of PBS (open square) or pTrc99A/BL21 cell homogenate extract (closed circle) in Example 2. Furthermore, the effect can be also apparent by comparing the S/N ratio of the crude extract of BL2 introduced with TM2/pTrc99A with the S/N ratio of PBS or the crude cell homogenate extract of BL2 introduced with pTrc99A shown in Table 3 in. Example 3.

### II. Method of Detection of the Present Invention (Embodiment 2)

The method of detection (Embodiment 2) according to the present invention relates to a method for detecting a substance in a biological sample and comprises:

1) providing a carrier on which biotin is bound and providing a fusion protein between a protein that specifically binds to a substance to be detected and a biotin-binding protein;

2) binding the fusion protein to the carrier provided in step 1) through binding between biotin and the biotin-binding protein to produce a fusion protein-bound carrier;

3) putting a biotin-binding protein into contact with the fusion protein-bound carrier produced in step 2) to block the carrier;

4) after the blocking step in step 3), adding a biological sample to the fusion protein-bound carrier; and

5) detecting a substance to be detected that has bound to the protein that specifically binds to the substance in the fusion protein.

In Embodiment 2 of the present invention, the method preparing the fusion protein-bound carrier in steps 1 and 2 is the same as that in Embodiment 1. In addition, the last step of detecting a substance to be detected that has been bound to a protein specifically binding to a substance to be detected in the fusion protein (step 4 of Embodiment 1 and step 5 of Embodiment 2) is the same as that in Embodiment 1.

The method of Embodiment 2 is characterized by a step of blocking a carrier (step 3 of Embodiment 2) before addition of a biological sample to the carrier.

In the present invention, background signals can be further suppressed by putting a biotin-binding protein into contact with a fusion protein-bound carrier before the addition of a biological sample to the carrier. It is believed that the biotin-binding protein binds to the biotin moiety of the fusion protein in a free state without binding on the carrier surface (FIG. 1), though not necessarily restricted by a theory.

The biotin-binding protein that will be put into contact with the binding carrier may be the same as or different from the biotin-binding protein constituting the fusion protein, and may be either a wild type or a variant. The biotin-binding activity of the variant may be equivalent, higher or lower compared to that of the wild type, but is preferably equivalent or lower. The variant may be, for example, but not limited to, a protein that has a biotin-binding activity lower than that of the wild-type protein and has a dissociation constant (KD) with biotin of 10⁻⁵ (M) or less. Such a biotin-binding protein can be produced by chemical modification (U.S. Pat. No. 5,051,356), but a method of modifying an amino acid sequence is most preferred (Qureshi et al., (2002), J. Biol. Chem., 276: 46422-46428).

In the present invention, the biotin-binding protein may be put into contact with the binding carrier by any method without particular limitation. That is, the biotin-binding protein may be added to a carrier directly or in a form of an appropriate solution. Furthermore, a blocking agent well-known to persons skilled in the art, such as BSA or casein, may be simultaneously used.

In a nonlimiting example, a biotin-binding protein is added to a desired buffer (e.g., TBS or PBS containing 0.02% to 1%, preferably 0.1% to 0.5%, of Tween-20 or Triton) in a final concentration of 1 to 500 μg/mL, preferably 10 to 100 μg/mL. BSA or casein may be further added thereto in a final concentration of 1 to 50 μg/mL, preferably 5 to 10 μg/mL. This blocking solution is put into contact with a carrier to which a biotin-binding protein fusion protein is bound by avidin-biotin binding at 10° C. to 40° C., preferably 20° C. to 30° C., for 10 min to 2 hr, preferably 30 min to 1 hr.

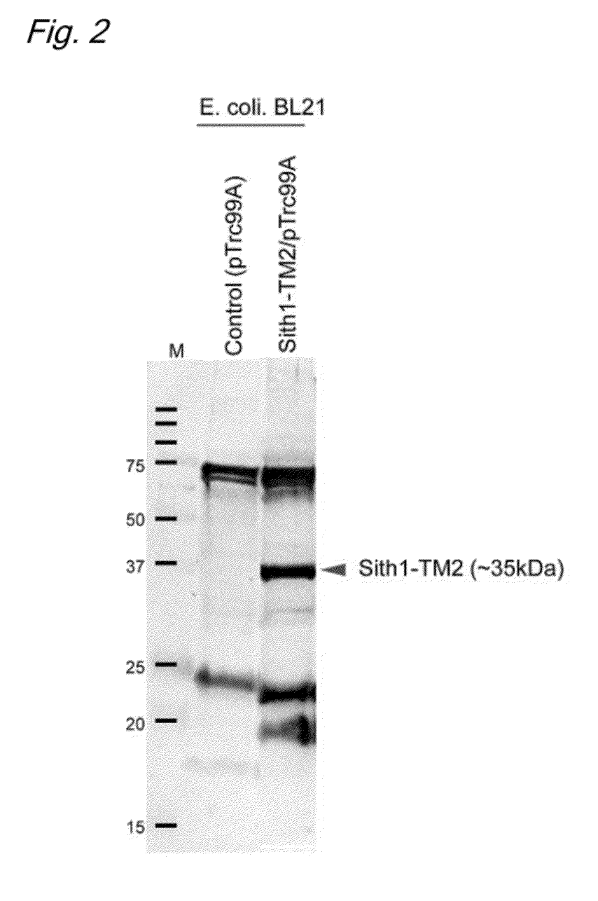

The effect of Embodiment 2 of the present invention can be apparent by, for example, in Example 2, comparing the value at antibody being 0 with the value in pTrc99A/BL21 cell homogenate extract (closed circle) shown in FIGS. 3-1 and 3-2. At antibody being 0, no antibody is present; hence, the fluorescence would be 0 if non-specific binding does not occur. Here, the comparison of the values at antibody being 0 shown by closed circles in FIGS. 3-1 and 3-2 confirms that the fluorescence value is reduced to almost a half in the case of blocking of a carrier with TM2 and is apparent that the non-specific binding is considerably reduced. In FIG. 3-2 showing the result of blocking of a carrier with TM2, the graph overall shifts below compared to that in FIG. 3-1. Furthermore, the effect can be apparent by comparing the value in “blocking by BSA” and the value in “blocking by BSA and TM2” for the results of pTrc99A/BL21 cell homogenate extract in Table 2 of Example 2.

### III. Method of Detection of the Present Invention (Embodiment 3)

The method of detection (Embodiment 3) according to the present invention relates to a method for detecting a substance in a biological sample and comprises:

1) providing a carrier on which biotin is bound and providing a fusion protein between a protein that specifically binds to a substance to be detected and a biotin-binding protein;

2) binding the fusion protein to the carrier provided in step 1) through binding between biotin and the biotin-binding protein to produce a fusion protein-bound carrier;

3) putting a biotin-binding protein into contact with the fusion protein-bound carrier produced in step 2) to block the carrier;

4) after the blocking step in step 3), mixing

(a) a biological sample, and

(b-i) a cell homogenate extract prepared from cells of the same species as the host cells used for expressing the fusion protein in step 1), and a biotin-binding protein, or

(b-ii) a cell homogenate extract prepared from cells of the same species as the host cells used for expressing the fusion protein in step 1), and genetically engineered to express a biotin-binding protein, and adding the mixture to the fusion protein-bound carrier; and

5) detecting a substance to be detected that has bound to the protein that specifically binds to the substance in the fusion protein.

Embodiment 3 is a combination of Embodiment 1 and Embodiment 2. Specifically, the addition of a cell homogenate extract and a biotin-binding protein in Embodiment 1 and the blocking of a carrier with a biotin-binding protein in Embodiment 2 are performed.

The effect of Embodiment 3 of the present invention can be apparent by, for example, comparing the results of TM2/pTrc99A/BL21 cell homogenate extract (closed triangle) in FIG. 3-2 with other results in FIGS. 3-1 and 3-2, for example, the results of PBS (open square) in Example 2. The effect can also be apparent by comparing the S/N ratio of the crude extract of BL21 introduced with TM2/pTrc99A with other results, for example, the S/N ratio of PBS, E. coli (BL21), or the crude cell homogenate extract of BL21 introduced with pTrc99A shown in Table 5 in Example 4.

### IV. Fusion Protein-Bound Carrier

The present invention also provides a carrier for detecting a substance in a biological sample. The carrier of the present invention is characterized by a fusion protein between a protein that specifically binds to a substance to be detected and a biotin-binding protein is bound to a carrier by binding between biotin and the biotin-binding protein, wherein the fusion protein is bound to the carrier by a method comprising:

1) providing a carrier on which biotin is bound and providing a fusion protein between a protein that specifically binds to a substance to be detected and a biotin-binding protein;

2) binding the fusion protein to the carrier provided in step 1) through binding between biotin and the biotin-binding protein to produce a fusion protein-bound carrier; and

3) putting a biotin-binding protein into contact with the fusion protein-bound carrier produced in step 2) to block the carrier. The carrier of the present invention is blocked with a biotin-binding protein before the addition of a biological sample.

Accordingly, in the carrier of the present invention, preferably, a fusion protein between a protein that specifically binds to a substance to be detected and a biotin-binding protein is bound to a carrier by avidin-biotin binding, and a biotin-binding protein is bound to unreacted biotin on the carrier by avidin-biotin binding.

### VII. Kit

The present invention also provides a kit for detecting a substance in a biological sample. The kit of the present invention comprises:

A) a carrier on which a fusion protein between a protein that specifically binds to a substance to be detected and a biotin-binding protein is bound by binding between biotin and the biotin-binding protein; and

an agent for diluting a biological sample, comprising

B-i) a cell homogenate extract prepared from cells of the same species as the host cells used for expressing the fusion protein in step A), and a biotin-binding protein, or

B-ii) a cell homogenate extract prepared from cells of the same species as the host cells used for expressing the fusion protein in step A), and genetically engineered to express a biotin-binding protein; and/or

C) a blocking agent containing a biotin-binding protein.

The agent for diluting a biological sample may be a cell homogenate extract (and biotin-binding protein) itself or may be an agent for diluting a cell homogenate extract together with a biological sample and may contain an appropriate buffer and a solvent such as a commercially available cell diluent or serum diluent.

### EXAMPLES

The present invention will now be described specifically by way of the following examples, which are not intended to limit the technical scope of the invention. Based on the descriptions of the specification, modifications and changes will be apparent to those skilled in the art, and such modifications and changes fall within the technical scope of the invention.

In Examples 1 and 2, a fusion protein between a human herpes virus 6 (HHV-6)-derived SITH-1 protein and tamavidin 2 was expressed in E. coli, and an E. coli crude extract obtained by sonication was directly reacted with a biotinylated microplate to thereby bind the fusion protein to the microplate through tamavidin-biotin binding. The resulting SITH-1 plate was reacted with human serum diluted with an E. coli crude extract (supplemented with rabbit anti-SITH-1 antibody; in this test, commercially available human sera supplemented with serial dilutions of rabbit anti-SITH-1 antibody (antiserum) were used as pseudo-clinical specimens) to measure the titer of anti-SITH-1 antibody contained in the human serum.

### Example 1

### SITH-1 Gene and Construction of Vector for Expression of Fusion Protein of Tamavidin 2

In this example, a gene encoding a fusion protein having tamavidin 2 (TM2) located at the C-terminal side of the SITH-1 via a linker was designed.

1-1. Design of Primer

In order to construct a SITH1-TM2 fusion gene, first, primers for fusion of SITH-1 and TM2 genes via a linker (5×linker: GGGGSGGGGSGGGGSGGGGSGGGGS) (SEQ ID NO: 18) were designed.

That is, a primer consisting of a DNA sequence encoding a C-terminal region of SITH-1 at the 5′-side, a linker at the center, and an N-terminal region of TM2 at the 3′-side (SITH1C-5×link-TM2N-F)(SEQ ID NO: 19) and a primer consisting of a DNA sequence encoding the N-terminal region of TM2 at the 5′-side, the linker, and the C-terminal region of SITH-1 at the 3′-side in the reverse direction (SITH1C-5×link-TM2N-R) (SEQ ID NO: 20) were designed.

Then, a primer consisting of a 5′ region including an N-terminal region of SITH-1 and an EcoRI restriction enzyme cleavage site (CCATGG) located upstream thereof (SITH1 5′ EcoRI-F (SEQ ID NO: 21)) and a primer consisting of a sequence encoding a 3′ region of the TM2 gene and a BamHI restriction enzyme cleavage site (GGATCC) located downstream thereof (TM2CtermBam) (SEQ ID NO: 22) were designed. The primers for construction of a fusion gene between SITH-1 and TM2 are summarized in Table 1.

TABLE 1
Primers for construction of a fusion gene between SITH-1 and TM2

| Name | Sequence | Length |
|---|---|---|
| SITH1 5′ EcoRI-F | AAAGAATTCGGATATGAAGAAAAAGTGTC | 29 mer |
| SITH1C-5xlink-TM2N-F | CCGAAAACTGAAATGAATGTGggtggcggtggcagc | 117 mer |
| | ggtggcggtggcagcggtggcggtggcagcggtggcggtggcagcgg | |
| | tggcggtggcagcTCAGACGTTCAATCTTCACTC | |
| SITH1C-5xlink-TM2N-R | GAGTGAAGATTGAACGTCTGAgctgccaccgccacc | 117 mer |
| | gctgccaccgccaccgctgccaccgccaccgctgccaccgccaccgct | |
| | gccaccgccaccCACATTCATTTCAGTTTTCGG | |
| TM2CtermBam | TTTGGATCCTTACTTCAACCTCGGTGCG | 28 mer |

The restriction enzyme recognition sites are underlined. The linker sequences are indicated in small letters.

1-2. PCR

In order to construct a SITH1-TM2 fusion gene, PCR was performed in two steps.

In the first step of PCR, a plasmid obtained by integrating the SITH-1 gene (ORF) (SEQ ID NO: 2) into FLAG Expression Vector (SIGMA) was used as a template to amplify a SITH-1 region with the primers SITH1 5′ EcoRI-F and SITH1C-5×link-TM2N-R, and a plasmid obtained by integrating the TM2 gene into vector pTrc99A (WO2002/072817) was used as a template to amplify a TM2 region with the primers SITH1C-5×link-TM2N-F and TM2CtermBam.

PCR was accomplished using a GeneAmp PCR System 9600 (PERKIN ELMER) in 20 μL reaction solution containing template DNA (500 ng), 10× ExTaq buffer (2 μL, TaKaRa), 2.5 mM dNTP (1.6 μL), primers (20 pmoles each), and 5 U/μL ExTaq (0.1 μL) under reaction conditions: 96° C. for 3 min, (95° C. for 1 min, 60° C. for 1 min, 72° C. for 2 min)×20 cycles, and 72° C. for 6 min. As a result, a PCR product of 579 by was obtained for the SITH-1 region, while a PCR product of 528 bp was obtained for the TM2 region. These PCR products were fractionated in a TAE buffer using agarose gel electrophoresis. The gel was cut out for each PCR product, and the products were collected using a QIAEX II gel extraction kit (QIAGEN). The extraction was performed according to the manual attached to the kit.

The two PCR products were used as templates to perform the second step of PCR using the primers SITH1 5′ EcoRI-F and TM2CtermBam. The reaction was accomplished using a GeneAmp PCR System 9600 (PERKIN ELMER) in 20 μL reaction solution containing template DNA (500 ng), 10× Pyrobest buffer (2 μL, TaKaRa), 2.5 mM dNTP (1.6 μL), primers (20 pmoles each), and 5 U/μL Pyrobest DNA polymerase (0.1 μL) under reaction conditions: 96° C. for 3 min, (95° C. for 1 min, 60° C. for 1 min, 72° C. for 2 min)×20 cycles, and 72° C. for 6 min. As a result, a PCR product of 990 bp was obtained.

1-3. Cloning

The SITH1-TM2 fusion gene obtained by PCR was cloned into vector pCR4Blunt TOPO (Invitrogen).

Specifically, first, ligation reaction was performed according to the manual attached to the vector kit. The DNA was introduced into E. coli TB1 by electroporation, and the plasmid DNA was extracted in a routine manner (Sambrook et al., 1989, Molecular Cloning, A laboratory manual, 2nd edition). Each plasmid for which the presence of an insert was confirmed was analyzed with M13 primers (TaKaRa) and an ABI PRISM fluorescent sequencer (Model 310 Genetic Analyzer, Perkin Elmer) to determine its nucleotide sequence, which was then confirmed to have no mutation in comparison with the sequence of the designed gene. The plasmid carrying the fusion gene was double-digested with EcoRI and BamHI, and agarose gel electrophoresis and purification were performed in the same manner as described above to collect a DNA fragment containing the fusion gene. This fragment was ligated into E. coli expression vector pTrc99A (Pharmacia), which had been digested with EcoRI and BamHI, using a Ligation Kit (TaKaRa). In the manner described above, a vector for SITH1-TM2 fusion protein expression, SITH1-TM2/pTrc99A, was completed. The ligation product was transformed into E. coli TB1, and the plasmid DNA was extracted and was further transformed into E. coli BL21. The resulting E. coli colonies were each used as a template to amplify an insert gene region by PCR with SITH1 5′ EcoRI-F and TM2CtermBam to thereby confirm the presence or absence of the insert gene.

In the manner described above, a vector SITH1-TM2/pTrc99A for expression of the fusion protein between SITH1 and TM2 was completed. The nucleotide sequence encoding SITH1-TM2 in the expression vector SITH1-TM2/pTrc99A is shown in SEQ ID NO: 23, and the amino acid sequence encoded thereby is shown in SEQ ID NO: 24.

In the 5′ side of SITH1-TM2, two amino acid residues are added downstream of the translation initiation methionine (nucleotides 4-9 in SEQ ID NO: 23, amino acid residues 2-3 in SEQ ID NO: 24) because the EcoRI site was used for integration into pTrc99A. These amino acid residues are followed by the SITH-1 sequence (nucleotides 10-483 in SEQ ID NO: 23, amino acid residues 4-161 in SEQ ID NO: 24), 5×linker (nucleotides 484-558 in SEQ ID NO: 23, amino acid residues 162-186 in SEQ ID NO: 24), and the TM2 sequence (excluding Met) (nucleotides 559-981 in SEQ ID NO: 23, amino acid residues 187-326 in SEQ ID NO: 24).

1-4. E. coli Expression

E. coli BL21 carrying SITH1-TM2/pTrc99A was inoculated into LB medium (50 mL) containing an antibiotic, ampicillin, (final concentration: 100 μg/mL) and cultured with shaking at 30° C. until the absorbance at OD600 reached 0.5. Then, 1 mM IPTG was added thereto, and shake culture was performed at 30° C. for an additional 5 hr. The cultured solution (50 mL) was centrifuged to collect the cells. The cells were suspended in 3 mL of 0.1 M HEPES/KOH (pH 7.4) and then homogenized by ultrasonication. The homogenate was centrifuged (15000 rpm), and the resulting supernatant was used as an E. coli crude extract. To confirm the expression of the TM2-fused SITH-1 protein, proteins contained in each crude extract were fractionated by SDS-PAGE and analyzed by Western blotting.

For detection of SITH-1, rabbit anti-SITH-1 antibody (i.e., anti-SITH-1 antibody (antiserum) prepared from rabbits immunized with a purified product of SITH-1 expressed in E. coli cells) and alkaline phosphatase-labeled anti-rabbit IgG antibody (BIO RAD) were each diluted 1000-fold and used. The results are shown in FIG. 2. A band of approximately 35 kDa was detected from the TM2-fused SITH-1-expressing E. coli cells. This size was substantially equal to the molecular weight (34.8 kDa) of TM2-fused SITH-1. It should be noted that E. coli crude extracts were also prepared in the same manner as shown above from E. coli BL21 carrying pTrc99A or TM2/pTrc99A (Takakura et al., (2009), FEBS J., 276: 1383-1397) after being cultured in the presence of IPTG.

### Example 2

### ELISA Detection of Anti-SITH-1 Antibody in Human Serum

The TM2-fused SITH-1-expressing E. coli crude extract obtained in Example 1 was diluted with 0.1 M HEPES/KOH (pH 7.4) into a total soluble protein concentration of 2 mg/mL, 100 μL of which was then added to each well in a biotin plate (manufactured by Sumitomo Bakelite Co., Ltd). The plate was allowed to stand at room temperature for 1 hr to immobilize the TM2-fused SITH-1 protein onto the biotin plate through tamavidin-biotin binding. Then, each well in the plate was washed with 0.1% Tween 20-containing TBS buffer (TBST) three times, followed by addition of a 5 μg/mL BSA/TBST solution or 5 μg/mL purified TM2/5 μg/mL BSA/TBST solution in a volume of 250 μL per well. The plate was allowed to stand at room temperature for 1 hr to block each well. Then, each well was washed with TBST three times.

Next, human serum (Human Serum pool, Cosmo-Bio, Inc.) was diluted 100-fold with PBS or with the pTrc99A-carrying E. coli crude extract (5 mg total soluble protein/mL) or the TM2/pTrc99A-carrying E. coli crude extract (5 mg total soluble protein/mL) prepared in Example 1-4. To the resulting solution, rabbit anti-SITH-1 antibody (antiserum) was added in serial dilution to give a volume ratio of 0.002, 0.001, 0.0005, or 0.00025. These human serum dilutions were added in 100 μL volumes to the plate on which the TM2-fused SITH-1 was immobilized, followed by incubation at room temperature for 1 hr.

It is expected that the antibody titer in serum of the rabbit anti-SITH-1 antibody (antiserum) is about 50 times higher compared to the SITH-1 antibody titer in serum of a patient with a typical depression (mood disorder). Accordingly, pseudo sample for serum of a patient with a typical depression can be prepared by mixing 1/50 volume of rabbit anti-SITH-1 antibody (antiserum) to a commercially available human serum (of a healthy person). Accordingly, the dilution rates of the antibody as discussed above provide samples similar to those when serum of depression patients are used after about 10 to 80 folds dilutions.

As a control, a biotin plate onto which nothing was bound was categorized into sections of two types blocking treatment, i.e., a section blocked with only BSA described above and a section blocked with BSA and TM2. In the section blocked with a solution containing tamavidin, since a part of the tamavidin in the blocking solution specifically binds to biotin on the plate, it is believed that the same state as a tamavidin-immobilized plate can be obtained. Then, human serum was diluted 100-fold with the same PBS solution or the same pTrc99A-carrying or TM2/pTrc99A-carrying E. coli crude extract (5 mg total soluble protein/mL) as used above, followed by addition of serially diluted rabbit anti-SITH-1 antibody. The resulting dilutions were added in 100 μL volumes to the control plate and incubated at room temperature for 1 hr. The rabbit anti-SITH-1 antibody was reacted with TM2-fused SITH-1 in the human serum, and then the plate was washed with TBST three times.

Then, to detect each of the rabbit anti-SITH-1 antibody bound to SITH-1 immobilized on the plate through tamavidin and the human IgG in serum which is presumed to be bound non-specifically in each well, a mixture of horseradish peroxidase-labeled goat anti-rabbit IgG antibody and peroxidase-labeled goat anti-human IgG antibody, each of which was diluted 5000-fold with TBST, was added in a volume of 100 μL per well, and the plate was left to stand for 1 hr at room temperature. Then, each well was washed with TBST three times, and peroxidase activity was detected. The activity was measured as follows: To each well, SuperSignal ELISA Pico Chemiluminescent Substrate (PIERCE) was added in a volume of 100 μL and allowed to stand for 5 min at room temperature, followed by measuring the luminescence intensity with a plate reader Infinite M200 (TECAN).

It should be noted that the data also include a luminescence intensity value measured at each concentration of rabbit anti-SITH-1 antibody for the control section sample (i.e., the section wherein the TM2 plate on which TM2-fused SITH-1 was not immobilized, but which was blocked with BSA or with both BSA and TM2 and treated with human serum containing anti-SITH-1 antibody at each concentration). The value of the control section was subtracted from the luminescence intensity value of each TM2-fused SITH-1-immobilized section. The resulting value was defined as the detected amount of anti-SITH-1 antibody contained in the serum.

FIG. 3 shows the effect of addition of E. coli homogenate extract to serum and the effect of blocking by TM2 on non-specific binding. In FIG. 3, open squares indicate the section where serum was diluted with PBS 100 fold, closed circles indicate the section where serum was diluted with an E. coli homogenate extract having only an expression vector 100 fold, and closed triangles indicate the section where serum was diluted with a TM2 expressing E. coli homogenate extract 100 fold. The vertical axis (luminescence) of each graph represents the amount of the detected anti-SITH-1 antibody while the lateral axis represents the dilution ratio of the serially diluted anti-SITH-1 antibody.

FIG. 3-1 shows the results of detection of the anti-SITH-1 antibody amount in serum without blocking by a biotin-binding protein (TM2), where open squares indicate human serum diluted with PBS, closed circles indicate human serum diluted with a pTrc99A-carrying E. coli crude extract, and closed triangles indicate serum diluted with a TM2/pTrc99A-carrying E. coli crude extract.

FIG. 3-2 shows the results of detection of the anti-SITH-1 antibody amount in serum without blocking by a biotin-binding protein (TM2), where open squares indicate human serum diluted with PBS, closed circles indicate human serum diluted with a pTrc99A-carrying E. coli crude extract, and closed triangles indicate serum diluted with a TM2/pTrc99A-carrying E. coli crude extract.

Further, the S/N ratio was calculated by the equation shown below to compare the effects of blocking and each serum dilution.

S/N ratio=Detected amount of anti-SITH-1 antibody in a section containing anti-SITH-1 antibody at each concentration/Detected amount of anti-SITH-1 antibody in a section free from anti-SITH-1 antibody

The equation shows that the detection sensitivity is higher when S/N ratio is larger.

Table 2 shows the calculated results of S/N ratio in each section.

TABLE 2

| Dilution ratio of | | | |
|---|---|---|---|
| anti-SITH-1 | | pTrc99A/BL21 cell | TM2/pTrc99A/BL21 cell |
| antibody | PBS | homogenate extract | homogenate extract |
| | Blocking by BSA | | |
| 0.002 | 1.6 | 3.8 | 6.0 |
| 0.001 | 1.7 | 3.6 | 4.7 |
| 0.0005 | 1.5 | 2.6 | 1.6 |
| 0.00025 | 1.3 | 2.2 | 0.9 |
| 0 | 1.0 | 1.0 | 1.0 |
| | Blocking by BSA and TM2 | | |
| 0.002 | 1.7 | 5.0 | 14.3 |
| 0.001 | 1.6 | 4.7 | 14.1 |
| 0.0005 | 1.5 | 3.6 | 10.9 |
| 0.00025 | 1.3 | 2.9 | 5.5 |
| 0 | 1.0 | 1.0 | 1.0 |

The S/N ratio in each section is normalized by the value at a dilution ratio of the anti-SITH-1 antibody of 0 (the case where the anti-SITH-1 antibody is not added, S/N = 1).

The anti-SITH-1 antibody contained in the serum showed non-specific binding in most of the serum sections diluted with PBS, and a high level of luminescence intensity was also detected even in the sections free from anti-SITH-1 antibody. In contrast, in the serum sections diluted with the pTrc99A-carrying E. coli crude extract or with the TM2/pTrc99A-carrying E. coli crude extract, or in the serum sections diluted with the pTrc99A-carrying E. coli crude extract, luminescence intensity was significantly low in sections without addition of the SITH-1 antibody, and non-specific binding was dramatically reduced. The non-specific binding in the section diluted with the TM2/pTrc99A-carrying E. coli crude extract was particularly low (FIGS. 3-1 and 3-2). In this case, those blocked with TM2 together with BSA (BSA+TM2) showed higher quatitativity in antibody detection in, in particular, the low concentration (dilution ratio: 0.0005 or low) range than those blocked with BSA only (FIG. 3-2).

Furthermore, it was revealed that in the section to which the pTrc99A-carrying E. coli crude extract was added and in the section to which the TM2/pTrc99A-carrying E. coli crude extract was added, the S/N ratio was increased by blocking the BSA containing TM2 to allow detection of the anti-SITH-1 antibody with high sensitivity (Table 2). In particular, in the section to which the TM2/pTrc99A-carrying E. coli crude extract was added, the increase in S/N ratio was significant.

These results indicated that non-specific binding originating from the human serum is reduced by blocking a TM2-fused SITH-1-immobilized plate with a solution containing TM2 and diluting human serum with a TM2 expressing E. coli crude extract, thereby enabling the sensitive, stable, and quantitative detection of anti-SITH-1 antibody even at a very low concentration.

### Example 3

### ELISA Detection of Anti-SITH-1 Antibody in Human Serum Using Magnetic Beads

In this example, various serum diluents were studied for their effect on the detection of SITH-1 antibody at very low concentrations in the system using biotinylated magnetic beads on which TM2-fused SITH-1 was immobilized.

3-1. Preparation of TM2-Fused SITH-1-Immobilized Magnetic Beads

To 1 mL (30 mg beads/mL) of magnetic beads, Dynabeads M-270 Amine, (DynalBiotech), 1 mL of 10 mM NHS-Lc-Lc-Biotin (PIERCE) was added. By end-over-end mixing at room temperature for 30 min, biotin was covalently bonded to the magnetic beads through reaction between amino groups and NHS active ester groups. Then, the magnetic beads was washed twice with a 0.1% BSA/0.01% Tween 20/PBS solution, and finally suspended in PBS buffer. The resulting magnetic beads (30 mg beads/mL PBS) were used as biotinylated magnetic beads.

TM2-fused SITH-1 was expressed in E. coli cells in the same manner as shown in Example 1-4 above, and the E. coli crude extract thus prepared was diluted with 0.1 M HEPES/KOH (pH 7.4) to give a total soluble protein concentration of 5 mg/mL, to which the biotinylated magnetic beads were then added. By end-over-end mixing at room temperature for 1 hr, TM2-fused SITH-1 was immobilized on the magnetic beads through tamavidin-biotin binding. Then, the magnetic beads were washed three times with 0.2% Tween 20-containing TBS buffer (TTBS).

In addition, a crude extract of TM2-expressing E. coli cells (Takakura et al., (2009), FEBS J, 276: 1383-1397) was diluted with 0.1 M HEPES/KOH (pH 7.4) to give a total soluble protein concentration of 5 mg/mL, to which the biotinylated magnetic beads were then added. By end-over-end mixing at room temperature for 1 hr, TM2 was immobilized through tamavidin-biotin binding. After washing in the same manner as shown above, the TM2-immobilized magnetic beads thus completed (30 mg beads/mL PBS) were used for subtraction of non-specific binding inherent to serum samples.

3-2. ELISA Analysis Using Magnetic Beads

To commercially available human serum (Human Serum pool, Cosmo-Bio), the same rabbit anti-SITH-1 antibody as used in Example 2 was added in a 1/50 volume to prepare SITH-1 antibody-containing human serum. On the other hand, human serum free from SITH-1 antibody was used as a control.

The human serum or rabbit SITH-1 antibody-containing human serum was diluted 1000-fold with PBS or with the pTrc99A vector product-expressing E. coli crude extract or the TM2-expressing E. coli crude extract, each of which had been adjusted with 0.1 M HEPES/KOH (pH 7.4) to give a total soluble protein concentration of 5 mg/mL. To the resulting solution, BSA was further added at a final concentration of 2% (w/v). In Example 2 described above, human serum was diluted 100-fold and rabbit anti-SITH-1 antibody was then added at a volume ratio of 0.00025 to 0.002 relative to the diluted serum. However, in this Example 3-2, rabbit anti-SITH-1 antibody was added in a 1/50 volume (at a volume ratio of 0.02) to human serum and then diluted 1000-fold. Thus, the dilution factor of the rabbit SITH-1 antibody is 1/50000, which is the same as that of depression patient's serum diluted about 1000-fold.

To the serum dilutions thus prepared (1 mL each), the TM2-fused SITH-1-immobilized magnetic beads or the TM2-immobilized magnetic beads were added in 10 μL volumes and reacted by end-over-end mixing for 1 hr at room temperature, followed by washing three times with TBST. Horseradish peroxidase-labeled goat anti-rabbit IgG antibody (for detection of the rabbit anti-SITH-1 antibody bound to the SITH-1 antigen immobilized on the magnetic beads) and peroxidase-labeled goat anti-human IgG antibody (for detection of human IgG bound non-specifically to the magnetic beads) were each diluted 5000-fold with 2% BSA-containing TBST and then mixed. This peroxidase-labeled secondary antibody mixture was added in 1000 μL volumes to the magnetic beads and mixed end-over-end for 1 hr at room temperature. Then, the beads were further washed three times with TBST, and 100 μL of detection reagent (1 step ELISA ultraTMB, PIERCE) was added and reacted at room temperature for 1 min. One hundred microliter of 2 M sulfuric acid was added to stop the reaction, and the degree of color development (absorbance at 450 nm wavelength, A450) was measured with a plate reader Infinite M200 (TECAN). The measurement was performed in duplicate for each treatment section to determine a mean value.

For use as data, the A450 value measured for each serum diluent (PBS, the pTrc99A vector product-expressing E. coli crude extract, or the TM2-expressing E. coli crude extract) in the TM2-fused SITH-1-immobilized magnetic beads was processed by subtraction of the A450 value measured for the corresponding serum diluent in the TM2-immobilized magnetic beads. The results obtained are shown in Table 3.

TABLE 3
Effect of serum diluents on SITH-1 antibody detection in human serum

| | A450 | | |
|---|---|---|---|
| | Serum containing | Serum free | |
| | SITH-1 antibody | from SITH-1 | |
| Serum diluent | (S) | antibody (N) | S/N ratio |
| PBS | 0.64 | 0.25 | 2.6 |
| pTrc99A-carrying | 0.14 | 0.04 | 3.5 |
| BL21 crude extract | | | |
| TM2/pTrc99A-carrying | 0.22 | 0.02 | 11.0 |
| BL21 crude extract | | | |

As shown in Table 3, the A450 value of SITH-1 antibody in human serum (S) was highest in the case of using PBS as a serum diluent, while the A450 value in serum free from SITH-1 antibody (N) was also very high, thus indicating that the S/N ratio (i.e., the ratio of the degree of color development in human serum containing rabbit anti-SITH-1 antibody to the degree of color development in serum free from rabbit anti-SITH-1 antibody) was lowest in PBS.

In contrast, when the TM2-expressing E. coli crude extract was used as a serum diluent, the A450 value in serum free from SITH-1 antibody was lowest, and hence non-specific binding could be greatly suppressed. In addition, the S/N ratio was highest when the TM2-expressing E. coli crude extract was used as a serum diluent, and the value thereof was larger than 10. This indicated that in the use of the TM2-fused SITH-1-immobilized magnetic beads, the addition of the TM2-expressing E. coli crude extract to human serum leads to a reduction in non-specific binding and high-sensitive detection of anti-SITH-1 antibody.

If depression (mood disorder) is diagnosed by quantitative determination of the SITH-1 antibody, the human serum containing rabbit anti-SITH-1 antibody and the human serum free from rabbit anti-SITH-1 antibody used in this specification can be recognized as serum of a depression (mood disorder) patient and serum of a healthy person, respectively. As shown in Table 3 above, the value of depression patient (S) is 0.64, and the value of healthy person (N) is 0.25 in the case of using PBS as a serum diluent, while the value of depression patient (S) is 0.22, and the value of healthy person (N) is 0.02 in the case of using the TM2/pTrc99A-carrying BL21 crude extract as a serum diluent. It is predicted that in a large number of clinical samples (serum), some of the sera from healthy persons show high non-specific binding, and, in contrast, some of the sera from depression patients do not clearly show a high SITH-1 antibody titer. Accordingly, as a method for detecting these differences, it is desirable that the non-specific binding (N) be low as much as possible and that the specific binding (S) can be certainly detected. From these points of view, the S/N ratio in this example is an effective index for comparing effects of each serum diluent for example.

### Example 4

### ELISA Detection of Anti-Harpin Antibody in Human Serum Using Fusion Protein Between Harpin and Tamavidin

In this Example, a fusion protein between Harpin, which is a protein derived from a plant pathogenic bacteria (hrpZpss protein, Takakura et al., 2004, Physiol. Mol. Plant Pathol., 64: 83-89), and tamavidin 2 (TM2) was expressed in E. coli cells and was immobilized on a biotinylated microplate by tamavidin-biotin binding. The resulting Harpin plate was blocked with tamavidin 2 and was then reacted with serum (human serum containing a rabbit anti-Harpin antibody) diluted with each E. coli crude extract. Subsequently, the anti-Harpin antibody was detected using peroxidase-labeled goat anti-human IgG antibody and horseradish peroxidase-labeled goat anti-rabbit IgG antibody. The details will be described below.

4-1. Construction of Vector for Expression of Fusion Protein of Harpin and Tamavidin 2 and E. coli Expression

A gene encoding a fusion protein having a TM2 sequence located at the C-terminal side of Harpin via a linker was constructed by PCR. In order to construct a Harpin-TM2 fusion gene, primers for linking Harpin and TM2 genes via a linker (5×linker: GGGGSGGGGSGGGGSGGGGSGGGGS) (SEQ ID NO: 18) were designed. The designed primers are summarized in Table 4.

TABLE 4
Primers for production of a gene encoding a fusion protein
between Harpin and TM2

| Primer Name | Sequence |
|---|---|
| HarpinNtermEcoRI-F | AAA GAA TTC atg cag agt ctc agt ctt aa |
| HarpinC-5xlink-TM2N-R | GTG AAG ATT GAA CGT CTG Agc tgc cac cgc cac cgc tgc |
| | cac cgc cac cgc tgc cac cgc cac cgc tgc cac cgc cac cgc tgc cac |
| | cgc cac cGG CTG CAG CCT GAT TGC GGG |
| HarpinC-5xlink-TM2N-F | CCC GCA ATC AGG CTG CAG CCg gtg gcg gtg gca gcg gtg |
| | gcg gtg gca gcg gtg gcg gtg gca gcg gtg gcg gtg gca gcg gtg gcg |
| | gtg gca gcT CAG ACG TTC AAT CTT CAC |

As shown in Table 4, a primer consisting of a DNA sequence encoding a C-terminal region of Harpin at the 5′-side, a linker at the center, and an N-terminal region of TM2 at the 3′-side (HarpinC-5×link-TM2N-F, Table 4, SEQ ID NO: 27) and a primer consisting of a DNA sequence encoding the N-terminal region of TM2 at the 5′-side, the linker at the center, and the C-terminal region of Harpin at the 3′-side in the reverse direction (HarpinC-5×link-TM2N-R, Table 4, SEQ ID NO: 26) were designed. Furthermore, a primer for the N-terminal region of Harpin, HarpinNtermEcoRI-F (including EcoRI cleavage sequence at the 5′ end, Table 4, SEQ ID NO: 25) was designed.

In order to construct a Harpin-TM2 fusion gene, PCR was performed in two steps. In the first step of PCR, a Harpin region was amplified with the primers HarpinNtermEcoRI-F and HarpinC-5×link-TM2N-R using a plasmid (Takakura et al., 2004, Physiol. Mol. Plant Pathol., 64: 83-89) in which a Harpin gene (ORF) (SEQ ID NO: 28) (encoded amino acid sequence: SEQ ID NO: 29) was integrated in pCR2.1 Vector (Invitrogen) as a template. Separately, a TM2 region was amplified with the primers HarpinC-5×link-TM2N-F and TM2CtermBam using a plasmid obtained by integrating the TM2 gene into vector pTrc99A (WO2002/072817) as a template.

PCR was accomplished using a GeneAmp PCR System 9600 (PERKIN ELMER) in 20 μL reaction solution containing template DNA (100 ng), 10× ExTaq buffer (2 μL, TaKaRa), 2.5 mM dNTP (1.6 μL), primers (20 pmoles each), and 5 U/μL ExTaq (0.1 μL) under the reaction conditions: 25 cycles of 94° C., 60° C., and 72° C. for 30 sec each. As a result, a PCR product of about 1 kbp was obtained for the Harpin region, while a PCR product of about 0.5 kbp was obtained for the TM2 region. These PCR products were fractionated in a TAE buffer using agarose gel electrophoresis. The gel was cut out for each PCR product, and the products were collected using a QIAEX II gel extraction kit (QIAGEN). The extraction was performed according to the manual attached to the kit.

The two PCR products were used as templates to perform the second step of PCR using the primers HarpinNtermEcoRI-F and TM2CtermBam. The reaction was accomplished using a GeneAmp PCR System 9600 (PERKIN ELMER) in 20 μL reaction solution containing template DNA (100 ng), 10× Pyrobest buffer (2 TaKaRa), 2.5 mM dNTP (1.6 μL), primers (20 pmoles each), and 5 U/μL Pyrobest DNA polymerase (0.1 μL) under the reaction conditions: (94° C. for 30 sec, 60° C. for 1 min, 72° C. for 1.5 min)×25 cycles. As a result, a PCR product of about 1.5 kbp was obtained.

The Harpin-TM2 fusion gene obtained by PCR was cloned into vector pCR4Blunt TOPO (Invitrogen). After confirmation of the nucleotide sequence, the plasmid carrying the fusion gene was double-digested with EcoRI and BamHI, and agarose gel electrophoresis and purification were performed in the same manner as described above to collect a DNA fragment containing the fusion gene. This fragment was ligated into E. coli expression vector pTrc99A (Pharmacia), which had been digested with EcoRI and BamHI, using a Ligation Kit (TaKaRa). The ligation product was transformed into E. coli BL21 (DE3), and the nucleotide sequence was confirmed.

As described above, a vector Harpin-TM2/pTrc99A for expression of the fusion protein between Harpin and TM2 was completed. The nucleotide sequence encoding Harpin-TM2 is shown in SEQ ID NO: 30, and the amino acid sequence encoded thereby is shown in SEQ ID NO: 31.

E. coli BL21 (DE3) carrying Harpin-TM2/pTrc99A was inoculated into LB medium (50 mL) containing an antibiotic, ampicillin, (final concentration: 100 μg/mL) and cultured with shaking at 30° C. until the absorbance at OD600 reached 0.5. Then, 1 mM IPTG was added thereto, and shake culture was performed at 30° C. for an additional 5 hr. The cultured solution (50 mL) was centrifuged to collect the cells. The cells were suspended in 3 mL of 0.1 M HEPES/KOH (pH 7.4) and then homogenized by ultrasonication. The homogenate was centrifuged (15000 rpm), and the resulting supernatant was used as an E. coli crude extract. To confirm the expression of the TM2-fused Harpin protein, proteins contained in each crude extract were fractionated by SDS-PAGE and analyzed by Western blotting.

For detection of Harpin, rabbit anti-Harpin-1 antibody (Takakura et al., 2004, Physiol. Mol. Plant Pathol., 64: 83-89) and alkaline phosphatase-labeled anti-rabbit IgG antibody (BIO RAD) were each diluted 1000-fold and used.

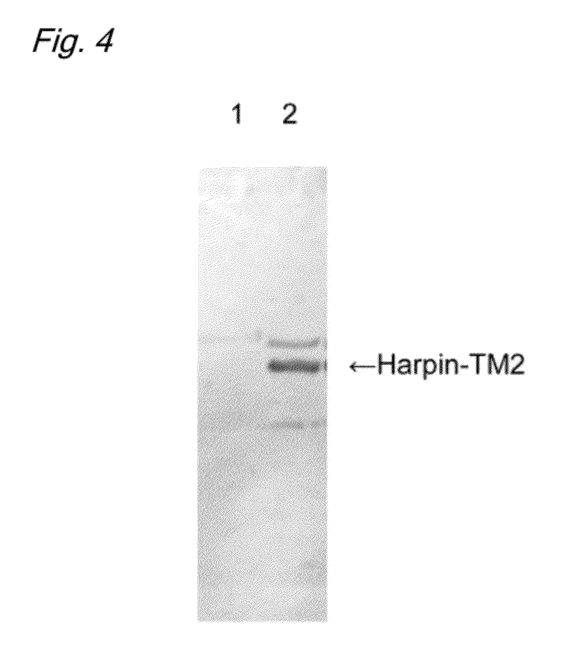

The results are shown in FIG. 4. In the E. coli homogenate extract having only an expression vector not containing an insert, no specific band was detected (FIG. 4, lane 1). In contrast, in the TM2-fused Harpin-expressing E. coli, a band migrating at approximately 50 kDa was detected (FIG. 4, lane 2). This size was substantially equal to the molecular weight (51.4 kDa) of TM2-fused Harpin.

4-2. ELISA Detection of Anti-Harpin Antibody in Human Serum

The TM2-fused Harpin-expressing E. coli crude extract was diluted with 0.1 M HEPES/KOH (pH 7.4) into a total soluble protein concentration of 1 mg/mL, 100 μL of which was then added to each well in a biotin plate (Sumitomo Bakelite Co., Ltd.). The plate was allowed to stand at room temperature for 1 hr for immobilization of the TM2-fused Harpin through tamavidin-biotin binding. Then, each well in the plate was washed with 0.1% Tween 20-containing TBS buffer (TBST) three times, followed by addition of a 50 μg/mL purified TM2 (WO2002/072817)/0.5% BSA/TBST solution in a volume of 250 μL per well. The plate was allowed to stand at room temperature for 1 hr to block each well. Then, each well was washed with TBST three times.

Next, human serum (Human Serum pool, Cosmo-Bio, Inc.) was diluted 100-fold with PBS or with the E. coli crude extract (5 mg total soluble protein/mL 0.1 M HEPES/KOH (pH 7.4)), the pTrc99A vector product-expressing E. coli crude extract (5 mg total soluble protein/mL 0.1 M HEPES/KOH (pH 7.4)), or the TM2-expressing E. coli crude extract (5 mg total soluble protein/mL 0.1 M HEPES/KOH (pH 7.4)). After addition of 1% BSA to the resulting solution, rabbit anti-Harpin antibody was added in a volume ratio of 1/500 thereto to prepare anti-Harpin antibody containing serum. Serum free from Harpin antibody was used as a control.

These sera (100 μL each) were added to the TM2-fused Harpin-immobilized plate and were allowed to stand for reaction at room temperature for 1 hr, followed by washing with TBST three times. A horseradish peroxidase-labeled goat anti-rabbit IgG antibody (for detection of the rabbit anti-Harpin antibody bound to the Harpin antigen) and a peroxidase-labeled goat anti-human IgG antibody (for detection of human IgG bound non-specifically to the wells) were each diluted 5000-fold with 1% BSA-containing TBST and then mixed. This peroxidase-labeled secondary antibody mixture was added in 100 μL volumes to the wells. The plate was allowed to stand for a reaction at room temperature for 1 hour. Then, the plate was further washed with TBST three times, and 100 μL detection reagent (1 step ELISA ultraTMB, PIERCE) was added and reacted at room temperature for 5 min. One hundred microliter of 2 M sulfuric acid was added to stop the reaction, and the degree of color development (absorbance at 450 nm wavelength, A450) was measured with a plate reader Infinite M200 (TECAN). The measurement was performed in triplicate for each treatment section to determine a mean value. In this example, the concentration of the antibody was relatively high, and the system was relatively low in non-specific binding. Consequently, subtraction of the measured value at the section where the antigen was not immobilized was unnecessary.

Table 5 shows the results.

TABLE 5
Effect of serum diluents on Harpin antibody detection in human serum

| | A450 | | |
|---|---|---|---|
| | Serum containing | Serum free | |
| | Harpin antibody | from Harpin | |
| Serum diluent | (S) | antibody (N) | S/N ratio |
| PBS | 2.26 | 0.23 | 9.7 |
| E. coli (BL21) crude | 2.13 | 0.19 | 11.3 |
| extract | | | |
| pTrc99A-carrying | 2.03 | 0.15 | 13.4 |
| BL21 crude extract | | | |
| TM2/pTrc99A-carrying | 2.16 | 0.12 | 16.8 |
| BL21 crude extract | | | |

As shown in Table 5, the A450 values (S) of Harpin antibody in human serum did not differ significantly among the serum diluents, whereas the values (N) in serum free from Harpin antibody as the control decreased in the order of the TM2/pTrc99A-carrying BL21 crude extract, the pTrc99A-carrying BL21 crude extract, the E. coli (BL21) crude extract, and PBS. This indicates that the TM2/pTrc99A-carrying BL21 crude extract could suppress non-specific binding in the highest degree. Regarding the S/N ratio (i.e., the degree of color development in human serum containing rabbit anti-Harpin antibody/the degree of color development in serum free from rabbit anti-Harpin antibody), the case of using the tamavidin 2-expressing E. coli extract (TM2/pTrc99A-carrying BL21 crude extract) as the serum diluent showed the highest value.

These results indicated that the dilution of human serum with an E. coli extract that has expressed tamavidin 2 suppresses non-specific binding to provide high-sensitive detection.

### SEQUENCE LISTING FREE TEXT

SEQ ID NO: 1: amino acid sequence of SITH-1

SEQ ID NO: 2: nucleotide sequence of SITH-1 ORF

SEQ ID NO: 3: nucleotide sequence of SITH-1 cDNA

SEQ ID NO: 4: nucleotide sequence of tamavidin 1

SEQ ID NO: 5: amino acid sequence of tamavidin 1

SEQ ID NO: 6: nucleotide sequence of tamavidin 2

SEQ ID NO: 7: amino acid sequence of tamavidin 2

SEQ ID NO: 8: example of linker sequence

SEQ ID NO: 9: example of linker sequence

SEQ ID NO: 10: example of linker sequence

SEQ ID NO: 11: example of linker sequence

SEQ ID NO: 12: example of linker sequence

SEQ ID NO: 13: example of linker sequence

SEQ ID NO: 14: example of linker sequence

SEQ ID NO: 15: example of linker sequence

SEQ ID NO: 16: example of linker sequence

SEQ ID NO: 17: example of linker sequence

SEQ ID NO: 18: example of linker sequence (used in Example)

SEQ ID NO: 19: nucleotide sequence of primer SITH1C-5×link-TM2N-F

SEQ ID NO: 20: nucleotide sequence of primer SITH1C-5×link-TM2N-R

SEQ ID NO: 21: nucleotide sequence of primer SITH1 5′ EcoRI-F

SEQ ID NO: 22: nucleotide sequence of primer TM2CtermBam

SEQ ID NO: 23: nucleotide sequence of SITH-1-TM2

SEQ ID NO: 24: amino acid sequence of SITH-1-TM2

SEQ ID NO: 25: nucleotide sequence of primer HarpinNtermEcoRI-F

SEQ ID NO: 26: nucleotide sequence of primer HarpinC-5×link-TM2N-R

SEQ ID NO: 27: nucleotide sequence of primer HarpinC-5×link-TM2N-F

SEQ ID NO: 28: nucleotide sequence of Harpin gene (ORF)

SEQ ID NO: 29: amino acid sequence encoded by Harpin gene (ORF)

SEQ ID NO: 30: nucleotide sequence of Harpin-TM2

SEQ ID NO: 31: amino acid sequence of Harpin-TM2

1. A method for detecting a substance in a biological sample, which comprises:
1) providing a carrier on which biotin is bound and providing a fusion protein between a protein that specifically binds to a substance to be detected and a biotin-binding protein;
2) binding the fusion protein to the carrier provided in step 1) through binding between biotin and the biotin-binding protein to produce a fusion protein-bound carrier;
3) mixing
(a) a biological sample, and
(b-i) a cell homogenate extract prepared from cells of the same species as the host cells used for expressing the fusion protein in step 1), and a biotin-binding protein, or
(b-ii) a cell homogenate extract prepared from cells of the same species as the host cells used for expressing the fusion protein in step 1), and genetically engineered to express a biotin-binding protein, and adding the mixture to the fusion protein-bound carrier produced in step 2); and
4) detecting a substance that has bound to the protein that specifically binds to a substance to be detected in the fusion protein.

2. A method for detecting a substance in a biological sample, which comprises:
1) providing a carrier on which biotin is bound and providing a fusion protein between a protein that specifically binds to a substance to be detected and a biotin-binding protein;
2) binding the fusion protein to the carrier provided in step 1) through binding between biotin and the biotin-binding protein to produce a fusion protein-bound carrier;
3) putting a biotin-binding protein into contact with the fusion protein-bound carrier produced in step 2) to block the carrier;
4) after the blocking step in step 3), adding a biological sample to the fusion protein-bound carrier; and
5) detecting a substance to be detected that has bound to the protein that specifically binds to the substance in the fusion protein.

3. A method for detecting a substance in a biological sample, which comprises:
1) providing a carrier on which biotin is bound and providing a fusion protein between a protein that specifically binds to a substance to be detected and a biotin-binding protein;
2) binding the fusion protein to the carrier provided in step 1) through binding between biotin and the biotin-binding protein to produce a fusion protein-bound carrier;
3) putting a biotin-binding protein into contact with the fusion protein-bound carrier produced in step 2) to block the carrier;
4) after the blocking step in step 3), mixing
(a) a biological sample, and
(b-i) a cell homogenate extract prepared from cells of the same species as the host cells used for expressing the fusion protein in step 1), and a biotin-binding protein, or
(b-ii) a cell homogenate extract prepared from cells of the same species as the host cells used for expressing the fusion protein in step 1), and genetically engineered to express a biotin-binding protein, and adding the mixture to the fusion protein-bound carrier; and
5) detecting a substance to be detected that has bound to the protein that specifically binds to the substance in the fusion protein.

4. The method according to claim 1 or 3, wherein step 3(b-i) in claim 1 or step 4(b-i) in claim 3 comprises adding a cell homogenate extract extracted from cells comprising any vector, as the cell homogenate extract.

5. The method according to any one of claims 1 to 3, wherein the biotin-binding protein is tamavidin or a variant thereof.

6. The method according to any one of claims 1 to 3, wherein the biological sample is selected from the group consisting of blood, serum, cerebrospinal fluid, saliva, sweat, urine, tear, lymph fluid, and mother's milk.

7. A carrier for detecting a substance in a biological sample, wherein
a fusion protein between a protein that specifically binds to a substance to be detected and a biotin-binding protein is bound to a carrier by binding between biotin and the biotin-binding protein, wherein
the fusion protein is bound to the carrier by a method comprising:
1) providing a carrier on which biotin is bound and providing a fusion protein between a protein that specifically binds to a substance to be detected and a biotin-binding protein;
2) binding the fusion protein to the carrier provided in step 1) through binding between biotin and the biotin-binding protein to produce a fusion protein-bound carrier; and
3) putting a biotin-binding protein into contact with the fusion protein-bound carrier produced in step 2) to block the carrier.

8. A kit for detecting a substance in a biological sample, which comprises:
A) a carrier on which a fusion protein between a protein that specifically binds to a substance to be detected and a biotin-binding protein is bound by binding between biotin and the biotin-binding protein; and
an agent for diluting a biological sample, comprising
B-i) a cell homogenate extract prepared from cells of the same species as the host cells used for expressing the fusion protein in step A), and a biotin-binding protein, or
B-ii) a cell homogenate extract prepared from cells of the same species as the host cells used for expressing the fusion protein in step A), and genetically engineered to express a biotin-binding protein; and/or
C) a blocking agent containing a biotin-binding protein.
